(19) United States
(12) Patent Application Publication
(10) Pub. No.: US 2017/0244034 A1
(43) Pub. Date: Aug. 24, 2017

(54) AROMATIC COMPOUND AND ORGANIC LIGHT-EMITTING DIODE INCLUDING THE SAME

(71) Applicant: National Tsing Hua University, Hsinchu City (TW)

(72) Inventors: Chien-Hong Cheng, Hsinchu City (TW); Yi-Hsiang Chen, Hsinchu City (TW); I-Ching Wu, Hsinchu City (TW)

(21) Appl. No.: 15/082,892

(22) Filed: Mar. 28, 2016

(30) Foreign Application Priority Data
Feb. 19, 2016 (TW) 105104865

(51) Int. Cl. H01L 51/00; C09K 11/02; C07C 211/54; C07D 403/10; C07C 211/58; C07D 209/86; C07D 209/88; C09K 11/06; C07C 211/56

(52) U.S. Cl. CPC H01L 51/006; C09K 11/06; C09K 11/02; C07C 211/54; C07C 211/56; C07C 211/58; C07D 209/86; H01L 51/0054; H01L 51/0072; C07D 209/88; C07D 403/10; H01L 51/0067; H01L 51/5028

(57) ABSTRACT

An aromatic compound represented by chemical formula 1 and an organic light-emitting diode including the same are provided.

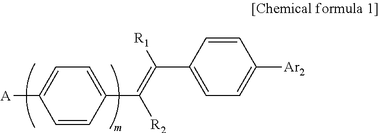

In chemical formula 1, A, Ar₂, R₁, R₂, and m are as described in the embodiments.

### CROSS-REFERENCE TO RELATED APPLICATION

This application claims the priority benefit of Taiwan application serial no. 105104865, filed on Feb. 19, 2016. The entirety of the above-mentioned patent application is hereby incorporated by reference herein and made a part of this specification.

### BACKGROUND OF THE INVENTION

Field of the Invention

The invention relates to a compound and an organic light-emitting diode including the same, and more particularly, to an aromatic compound and an organic light-emitting diode including the same.

Description of Related Art

An organic light-emitting diode (OLED) flat panel display has advantages such as wider viewing angle, faster reaction time, and smaller size in comparison to a liquid crystal display, and is currently applied in display having large area, high brightness, and full color.

To develop a flat panel display having full color, the development of a stable light-emitting material (red, green, blue) having high luminous efficiency is the main object of current OLED research. However, in comparison to a red light-emitting material and a green light-emitting material, the development of a blue light-emitting material in luminous efficiency and emission lifetime is slower, and therefore the development of a novel blue light-emitting material having high luminous efficiency and long life is an important current object.

### SUMMARY OF THE INVENTION

The invention provides an aromatic compound capable of achieving an organic light-emitting diode having high luminous efficiency and long life.

The invention provides an aromatic compound represented by the following chemical formula 1:

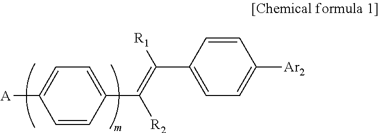

In chemical formula 1, R₁ and R₂ are each independently hydrogen, halogen, a C₁ to C₆ alkyl group, or an aromatic group, m is a integer of 0 or 1, A is a substituted or unsubstituted carbazole group Ar₁, or an organic amine group, and Ar₂ is a substituted or unsubstituted pyrenyl group, a substituted or unsubstituted sulfonyl group, a substituted or unsubstituted triazine group, or a substituted or unsubstituted

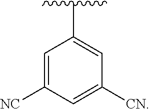 [SMILES: *c1cc(C#N)cc(C#N)c1]

In an embodiment of the invention, the above aromatic compound is represented by the following chemical formula 2:

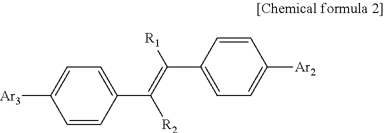

In chemical formula 2, Ar₃ is selected from the following structural formulas,

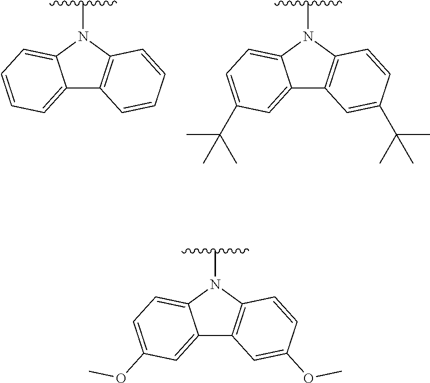

the remaining substituents are defined the same as in chemical formula 1.

In an embodiment of the invention, the above aromatic compound is represented by the following chemical formula 3:

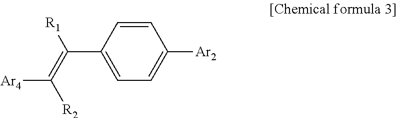

In chemical formula 3, Ar₄ is selected from the following structural formulas,

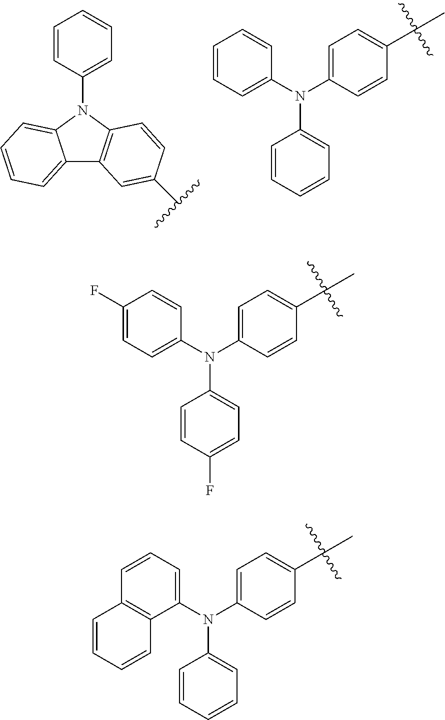 [SMILES: *c1ccc2c(c1)c1ccccc1n2-c1ccccc1 ; *c1ccc(N(c2ccccc2)c2ccccc2)cc1 ; *c1ccc(N(c2ccc(F)cc2)c2ccc(F)cc2)cc1 ; *c1ccc(N(c2ccccc2)c2cccc3ccccc23)cc1]

the remaining substituents are defined the same as in chemical formula 1.

In an embodiment of the invention, the above Ar₂ is selected from the following structural formulas,

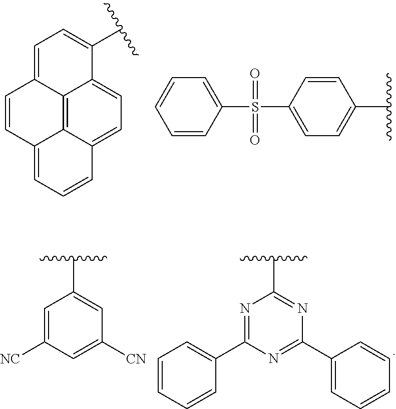

The invention provides an organic light-emitting diode including a cathode, an anode, and a light-emitting layer. The light-emitting layer is disposed between the cathode and the anode, wherein the light-emitting layer contains the above aromatic compound.

In an embodiment of the invention, the above organic light-emitting diode is, for instance, a blue light-emitting diode.

In an embodiment of the invention, the above light-emitting layer includes a host light-emitting material and a guest light-emitting material.

In an embodiment of the invention, the above host light-emitting material includes the aromatic compound.

In an embodiment of the invention, the above guest light-emitting material includes the aromatic compound.

In an embodiment of the invention, the above host light-emitting material is, for instance, 1-(2,5-dimethyl-4-(1-pyrenyl) phenyl)pyrene (DMPPP), 4,4′-N,N′-dicarbazole-biphenyl (CBP), or 2-(3-(pyren-1-yl)phenyl)triphenylene (m-PPT).

In an embodiment of the invention, the above organic light-emitting diode further includes at least one auxiliary layer, and the auxiliary layer is selected from the group consisting of a hole injection layer, a hole transport layer, a hole blocking layer, an electron injection layer, an electron transport layer, and an electron blocking layer.

In an embodiment of the invention, the above at least one auxiliary layer contains the above aromatic compound.

Based on the above, the aromatic compound of the invention has the characteristics of blue light emission, high quantum efficiency, and good thermal stability. Moreover, the aromatic compound of the invention can be applied in the light-emitting layer or the hole transport layer of an organic light-emitting diode to increase external quantum efficiency, maximum brightness, current efficiency, power efficiency, and life of the organic light-emitting diode.

In order to make the aforementioned features and advantages of the disclosure more comprehensible, embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a cross-sectional schematic of an organic light-emitting diode according to an embodiment of the invention.

FIG. 2 is a cross-sectional schematic of an organic light-emitting diode according to another embodiment of the invention.

FIG. 3A and FIG. 3B show transient light excitation fluorescence curves of a toluene solution containing the compound CZSSO under the introduction of air and nitrogen, respectively.

FIG. 4A and FIG. 4B show transient light excitation fluorescence curves of a toluene solution containing the compound TCZSSO under the introduction of air and nitrogen, respectively.

FIG. 5A and FIG. 5B show transient light excitation fluorescence curves of a toluene solution containing the compound OCZSSO under the introduction of air and nitrogen, respectively.

FIG. 6A and FIG. 6B show transient light excitation fluorescence curves of a toluene solution containing the compound CZSDCN under the introduction of air and nitrogen, respectively.

FIG. 7A and FIG. 7B show transient light excitation fluorescence curves of a toluene solution containing the compound CZSDPT under the introduction of air and nitrogen, respectively.

FIG. 8 shows transient light excitation fluorescence curves of the organic light-emitting diodes of experimental example 1 to experimental example 4.

FIG. 9 shows transient light excitation fluorescence curves of the organic light-emitting diodes of experimental example 5 to experimental example 7 and the comparative example.

FIG. 10 shows transient light excitation fluorescence curves of the organic light-emitting diodes of experimental example 8 to experimental example 10.

FIG. 11 shows a transient light excitation fluorescence curve of the organic light-emitting diode of experimental example 18.

FIG. 12 shows a brightness-external quantum efficiency curve of the organic light-emitting diodes of experimental example 11 to experimental example 15.

### DESCRIPTION OF THE EMBODIMENTS

In the following, embodiments of the invention are described in detail. However, the embodiments are exemplary, and the disclosure is not limited thereto.

The aromatic compound according to an embodiment of the invention is represented by the following chemical formula 1:

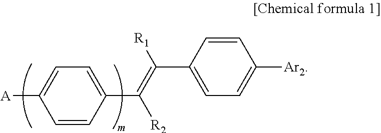

In chemical formula 1, R₁ and R₂ are each independently hydrogen, halogen, a C₁ to C₆ alkyl group, or an aryl group. m is an integer of 0 or 1. A is a substituted or unsubstituted carbazole group Ar₁ or an organic amine group. Ar₂ is a substituted or unsubstituted pyrenyl group, a substituted or unsubstituted sulfonyl group, a substituted or unsubstituted triazine group, or a substituted or unsubstituted

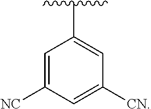 [SMILES: *c1cc(C#N)cc(C#N)c1]

In an embodiment of the invention, the aromatic compound is represented by the following chemical formula 2:

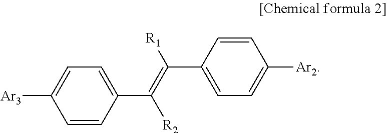

In chemical formula 2, R₁, R₂, and Ar₂ are defined the same in chemical formula 1, and Ar₃ is, for instance, selected from the following structural formulas:

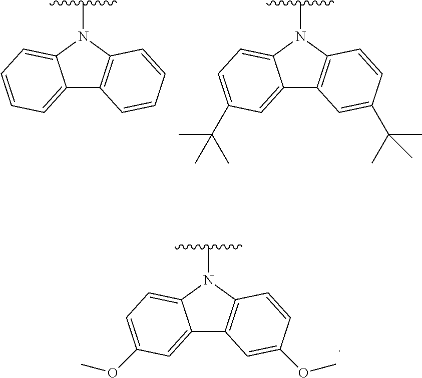

In another embodiment of the invention, the aromatic compound is represented by the following chemical formula 3:

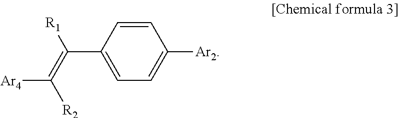

In chemical formula 3, R₁, R₂, and Ar₂ are defined the same in chemical formula 1, and Ar₄ is selected from the following structural formulas:

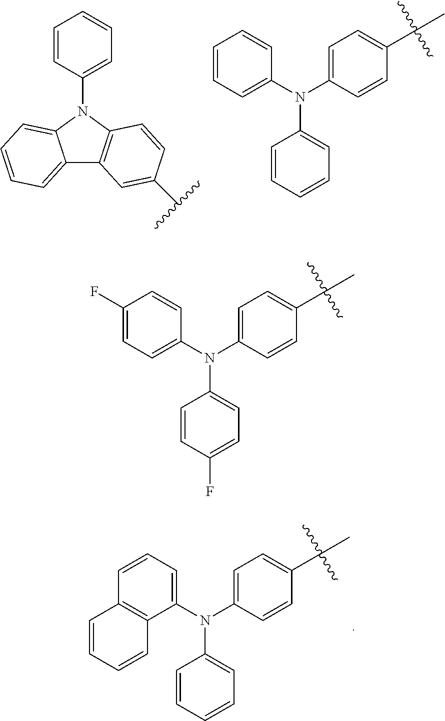 [SMILES: *c1ccc2c(c1)c1ccccc1n2-c1ccccc1 ; *c1ccc(N(c2ccccc2)c2ccccc2)cc1 ; *c1ccc(N(c2ccc(F)cc2)c2ccc(F)cc2)cc1 ; *c1ccc(N(c2ccccc2)c2cccc3ccccc23)cc1]

In the present specification, unless otherwise specified, the term “substituted” refers to substitution by the following groups: halogen, an aryl group, a hydroxyl group, an alkenyl group, a C₁ to C₂₀ alkyl group, an alkynyl group, a cyano group, a trifluoromethyl group, an alkylamino group, an amine group, a C₁ to C₂₀ alkoxy group, a heteroaryl group, an aryl group having a halogen substituent, an aralkyl group having a halogen substituent, an aryl group having a haloalkyl substituent, an aralkyl group having a haloalkyl substituent, a C₁ to C₂₀ alkyl group having an aryl substituent, a cycloalkyl group, an amine group having a C₁ to C₂₀ alkyl substituent, an amine group having a haloalkyl substituent, an amine group having an aryl substituent, an amine group having a heteroaryl substituent, a phosphinyloxy group having an aryl substituent, a phosphinyloxy group having a C₁ to C₂₀ alkyl substituent, a phosphinyloxy group having a haloalkyl substituent, a phosphinyloxy group having a halogen substituent, a phosphinyloxy group having a heteroaryl substituent, a nitro group, a carbonyl group, an arylcarbonyl group, a heteroarylcarbonyl group, or a C₁ to C₂₀ alkyl group having a halogen substituent.

In the present specification, the term “aryl group” refers to a substituent including a ring having a conjugate p orbital, and the aryl group can be a monocyclic, polycyclic, or fused ring polycyclic functional group.

Specifically, examples of the aryl group include a phenyl group, a phenylene group, a naphthyl group, a naphthylene group, a pyrenyl group, an anthryl group, and a phenanthryl group, but are not limited thereto.

In the present specification, the term “heteroaryl group” refers to an aryl group in a functional group including 1 to 3 heteroatoms selected from N, O, S, P, and Si and remaining carbon atoms. The heteroaryl group can be a fused ring, wherein each ring can include 1 to 3 heteroatoms.

Specifically, examples of the heteroaryl group include a furyl group, a furylene group, a fluorenyl group, a pyrrolyl group, a thienyl group, an oxazolyl group, an imidazolyl group, a thiazolyl group, a pyridyl group, a pyrimidinyl group, a quinazolinyl group, a quinolyl group, an isoquinolyl group, and an indolyl group, but are not limited thereto.

In the following, the organic light-emitting diode of an embodiment of the invention is described with reference to figures.

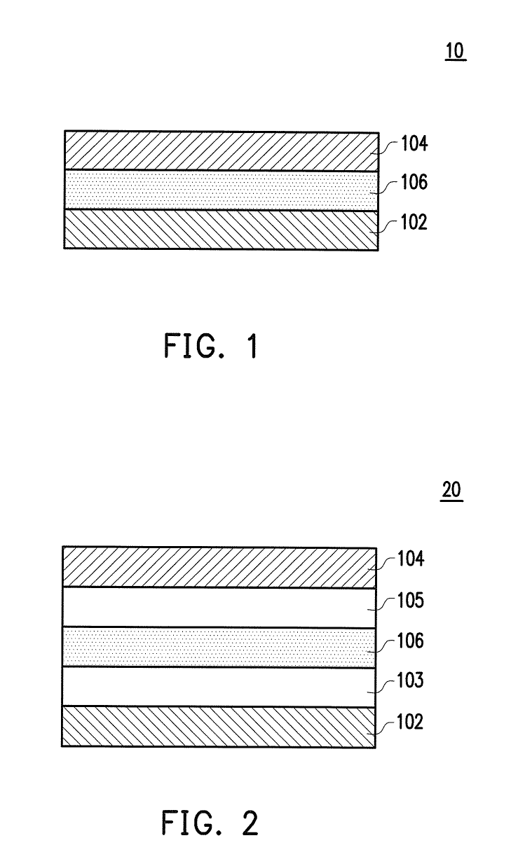

FIG. 1 is a cross-sectional schematic of an organic light-emitting diode according to an embodiment of the invention.

Referring to FIG. 1, an organic light-emitting diode 10 of the present embodiment includes an anode 102, a cathode 104, and a light-emitting layer 106. The light-emitting layer 106 is disposed between the anode 102 and the cathode 104. The anode 102 can be obtained from a conductor having high work function to facilitate the injection of holes in the light-emitting layer 106. The material of the anode 102 is, for instance, metal, metal oxide, a conducting polymer, or a combination thereof. Specifically, the metal is, for instance, nickel, platinum, vanadium, chromium, copper, zinc, gold, or an alloy thereof; the metal oxide is, for instance, zinc oxide, indium oxide, indium tin oxide (ITO), or indium zinc oxide (IZO); the combination of the metal and the oxide is, for instance, a combination of ZnO and Al or a combination of SnO₂ and Sb; the conductive polymer is, for instance, poly(3-methylthiophene), poly(3,4-(ethylene-1,2-dioxy)thiophene (PEDT), polypyrrole, or polyaniline, but the invention is not limited thereto.

The cathode 104 can be obtained from a conductor having low work function to facilitate the injection of electrons in the light-emitting layer 106. The material of the cathode 104 is, for instance, metal or multilayer structure material. Specifically, the metal is, for instance, magnesium, calcium, sodium, potassium, titanium, indium, yttrium, lithium, gadolinium, aluminum, silver, tin, lead, cesium, barium, or an alloy thereof; the material of the multilayer structure is, for instance, LiF/Al, LiO₂/Al, LiF/Ca, LiF/Al, or BaF₂/Ca, but the invention is not limited thereto.

In the present embodiment, the light-emitting layer 106 includes the aromatic compound of the above embodiments. Specifically, the light-emitting layer 106 includes one aromatic compound of the above embodiments, at least two aromatic compounds of the above embodiments, or a mixture of at least one of the aromatic compounds of the above embodiments and other compounds.

The light-emitting layer 106 generally includes a host light-emitting material and a guest light-emitting material. The aromatic compound of the above embodiments can be mixed with the host light-emitting material as the guest light-emitting material, and can also be mixed with the guest light-emitting material as the host light-emitting material.

Other host light-emitting materials include, for instance, a condensation aromatic cycle derivative, a heterocycle-containing compound, or a similar compound thereof. The condensation aromatic cycle derivative is, for instance, an anthracene derivative, a pyrene derivative, a naphthalene derivative, a pentacene derivative, a phenanthrene derivative, a fluoranthene compound, or a similar compound thereof. The heterocycle-containing compound is, for instance, a carbazole derivative, a dibenzofuran derivative, a ladder-type furan compound, a pyrimidine derivative, or a similar compound thereof. Specifically, the host light-emitting material is, for instance, 1-(2,5-dimethyl-4-(1-pyrenyl) phenyl)pyrene (DMPPP), 4,4′-N,N′-dicarbazole-biphenyl (CBP), or 2-(3-(pyren-1-yl)phenyl)triphenylene (m-PPT), but the invention is not limited thereto.

The guest light-emitting material other than the aromatic compound of the above embodiments is, for instance, an arylamine derivative, a styrylamine compound, a boron complex, a fluoranthene compound, a metal complex, or a similar compound thereof. Specifically, the arylamine derivative is, for instance, a fused aromatic ring derivative substituted by an arylamine group, and examples thereof include, for instance, pyrene, anthracene, chrysene, and periflanthene having an arylamine group; specific examples of the styrylamine compound include styrylamine, styryldiamine, styryltriamine, and styryltetramine. Examples of the metal complex include an iridium complex and a platinum complex, but are not limited thereto.

In an embodiment, the organic light-emitting diode 10 further includes at least one auxiliary layer, and the auxiliary layer is selected from the group consisting of a hole injection layer, a hole transport layer, a hole blocking layer, an electron injection layer, an electron transport layer, and an electron blocking layer.

In an embodiment, at least one auxiliary layer contains the aromatic compound of the above embodiments.

FIG. 2 is a cross-sectional schematic of an organic light-emitting diode according to another embodiment of the invention. In FIG. 2, the same elements as FIG. 1 are represented by the same reference numerals, and descriptions of the same technical content are omitted. An organic light-emitting diode 20 includes an anode 102, a hole transport layer 103, a light-emitting layer 106, an electron transport layer 105, and a cathode 104.

In the present embodiment, the light-emitting layer 106 includes the aromatic compound of the above embodiments. In another embodiment, in addition to the light-emitting layer 106, at least one of the hole transport layer 103 and the electron transport layer 105 also includes the aromatic compound of the above embodiments.

In the following, the above embodiments are described in more detail with reference to examples. However, the examples are not to be construed as limiting the scope of the invention in any sense.

Synthesis of Organic Compound

[Synthesis of Intermediate Product]

### Synthesis Example 1: Synthesis of Intermediate Product I-1

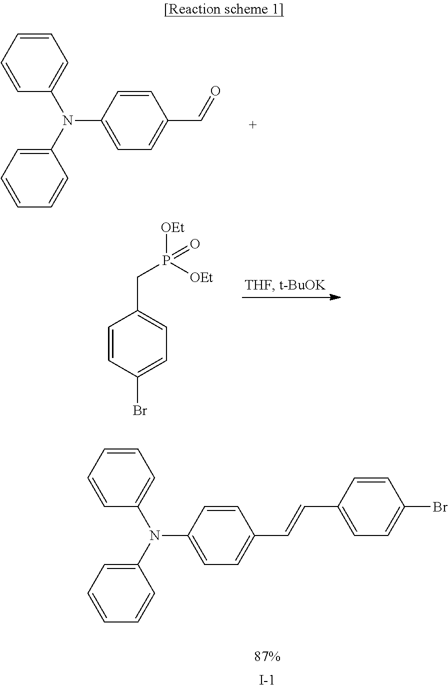

4-(diphenylamino)benzaldehyde (2.73 g, 10.0 mmol) and diethyl (4-bromobenzyl)phosphonate (3.53 g, 11.5 mmol) were placed in a two-neck bottle, and after the bottle was vacuumed and nitrogen was introduced, 20 mL of anhydrous tetrahydrofuran (THF) was added; in an ice bath, t-BuOK (3.36 g, 30 mmol) dissolved in THF (30 mL) was slowly added to mix, and the mixture was reacted at 0° C. for 15 minutes. The solvent was removed via concentration under reduced pressure, and then purification was performed via column chromatography (n-hexane:dichloromethane=9:1) to obtain a yellow intermediate product I-1 ((E)-4-(4-bromostyryl)-N,N-diphenylaniline) (3.71 g, yield: 87%).

¹H NMR (400 MHz, CDCl3, δ): 7.45 (d, J=8.4 Hz, 2H), 7.36 (d, J=8.8 Hz, 2H), 7.34 (d, J=8.8 Hz, 2H), 7.28-7.24 (m, 4H), 7.11 (d, J=7.6 Hz, 4H), 7.05-7.01 (m, 5H), 6.90 (d, J=16 Hz, 1H).

¹³C NMR (100 MHz, CDCl₃, δ): 147.53, 147.38, 136.50, 131.65, 130.89, 129.68, 129.26, 127.69, 127.37, 125.55, 124.52, 123.30, 123.09, 120.80.

HRMS (m/z): [M]⁺ calcd for C₂₆H₂₀BrN, 425.0779. found, 425.0772.

### Synthesis Example 2: Synthesis of Intermediate Product I-2

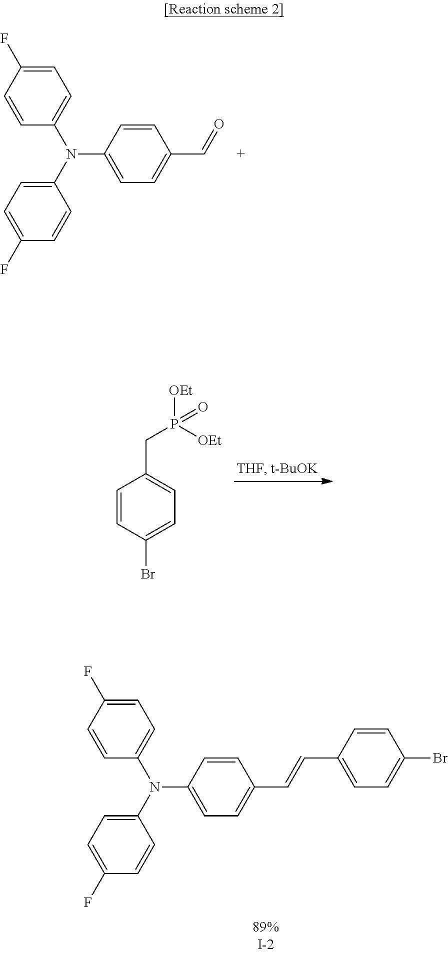

4-(bis(4-fluorophenyl)amino)benzaldehyde (4.64 g, 15 mmol) and diethyl (4-bromobenzyl)phosphonate (5.07 g, 16.5 mmol) were placed in a two-neck bottle, and after the bottle was vacuumed and nitrogen was introduced, 20 mL of anhydrous tetrahydrofuran (THF) was added; in an ice bath, t-BuOK (5.0 g, 45 mmol) dissolved in THF (30 mL) was slowly added to mix, and the mixture was reacted at 0° C. for 15 minutes. The solvent was removed via concentration under reduced pressure, and then purification was performed via column chromatography (n-hexane:dichloromethane=9:1) to obtain a yellow intermediate product I-2 ((E)-4-(4-bromostyryl)-N,N-bis(4-fluorophenyl)aniline) (6.17 g, yield: 89%).

¹H NMR (400 MHz, CDCl₃, δ): 7.44 (d, J=8.4 Hz, 2H), 7.33 (d, J=8.8 Hz, 2H), 7.32 (d, J=8.8 Hz, 2H), 7.06-6.92 (m, 11H), 6.88 (d, J=16 Hz, 1H).

¹³C NMR (100 MHz, CDCl₃, δ): 158.00 ppm (d, ¹³C-¹⁹F coupling J=242 Hz, C), 147.62 (C), 143.42 (d, ¹³C-¹⁹F coupling J=3 Hz, C), 136.47 (C), 131.69 (CH), 130.69 (C), 128.70 (CH), 127.71 (CH), 127.47 (CH), 126.23 (d, ¹³C-¹⁹F coupling J=7.6 Hz, CH), 125.62 (CH), 122.10 (CH), 120.86 (C), 116.15 (d, ¹³C-¹⁹F coupling J=22.8 Hz, CH)

HRMS (m/z): [M]⁺ calcd. for C₂₆H₁₈BrF₂N, 461.0591. found, 461.0594.

### Synthesis Example 3: Synthesis of Intermediate Product I-3

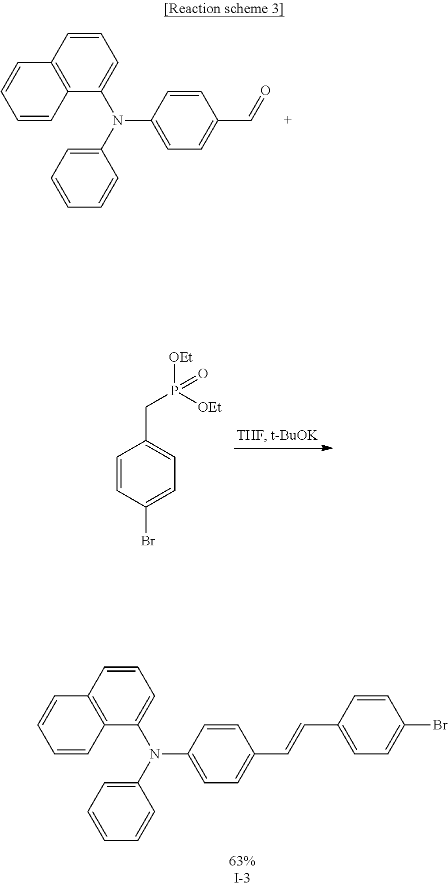

4-(naphthalen-1-yl(phenyl)amino)benzaldehyde (2.87 g, 8.9 mmol) and diethyl (4-bromobenzyl)phosphonate (3.0 g, 9.76 mmol) were placed in a two-neck bottle, and after the bottle was vacuumed and nitrogen was introduced, 20 mL of anhydrous tetrahydrofuran (THF) was added; in an ice bath, t-BuOK (2.24 g, 20 mmol) dissolved in THF (30 mL) was slowly added to mix, and the mixture was reacted at 0° C. for 15 minutes. The solvent was removed via concentration under reduced pressure, and then purification was performed via column chromatography (n-hexane:dichloromethane=9:1) to obtain a yellow intermediate product I-3 ((E)-N-(4-(4-bromostyryl)phenyl)-N-phenylnaphthalen-1-amine) (2.67 g, yield: 63%).

¹H NMR (400 MHz, CDCl₃, δ): 7.91-7.86 (m, 2H), 7.77 (d, J=8.0 Hz, 1H), 7.48-7.29 (m, 10H), 7.22-6.94 (m, 8H), 6.85 (d, J=16 Hz, 1H).

¹³C NMR (100 MHz, CDCl₃, δ): 148.22, 147.89, 143.11, 136.63, 135.24, 131.67, 131.11, 129.95, 129.17, 128.93, 128.41, 127.66, 127.37, 127.24, 126.66, 126.48, 126.34, 126.18, 125.13, 124.11, 122.48, 122.26, 121.11, 120.69.

HRMS (m/z): [M]⁺ calcd. for C₃₀H₂₂BrN, 475.0936. found, 475.0937.

### Synthesis Example 4: Synthesis of Intermediate Product I-4

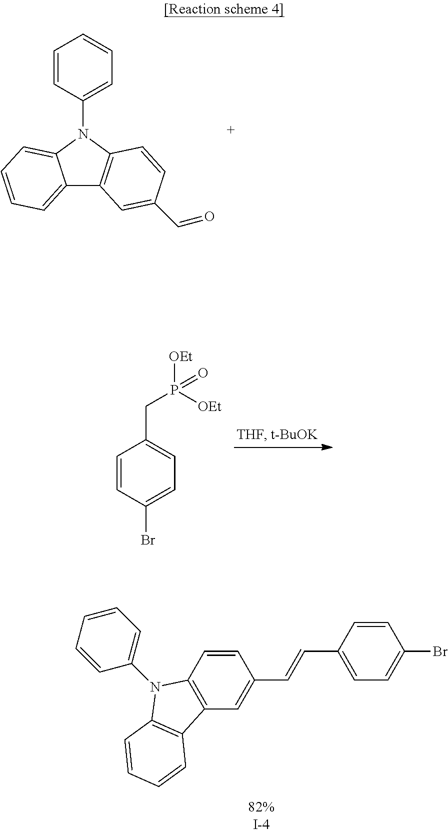 [SMILES: O=Cc1ccc2c(c1)c1ccccc1n2-c1ccccc1 ; CCOP(=O)(Cc1ccc(Br)cc1)OCC ; Brc1ccc(/C=C/c2ccc3c(c2)c2ccccc2n3-c2ccccc2)cc1]

9-phenyl-9H-carbazole-3-carbaldehyde (3.52 g, 13 mmol) and diethyl (4-bromobenzyl)phosphonate (4.42 g, 14.4 mmol) were placed in a two-neck bottle, and after the bottle was vacuumed and nitrogen was introduced, 20 mL of anhydrous tetrahydrofuran (THF) was added; in an ice bath, t-BuOK (3.36 g, 30 mmol) dissolved in THF (30 mL) was slowly added to mix, and the mixture was reacted at 0° C. for 15 minutes. The solvent was removed via concentration under reduced pressure, and then purification was performed via column chromatography (n-hexane:dichloromethane=5:1) to obtain a white intermediate product I-4 ((E)-3-(4-bromostyryl)-9-phenyl-9H-carbazole) (4.52 g, yield: 82%).

¹H NMR (400 MHz, CDCl₃, δ): 8.26 (s, 1H), 8.15 (d, J=7.6 Hz, 1H), 7.60-7.35 (m, 13H), 7.31-7.27 (m, 2H), 7.07 (d, J=16.4 Hz, 1H).

¹³C NMR (100 MHz, CDCl₃, δ): 141.26, 140.64, 137.41, 136.76, 131.68, 130.11, 129.88, 129.18, 127.68, 127.53, 126.96, 126.18, 125.04, 124.68, 123.72, 123.24, 120.61, 120.33, 120.17, 118.64, 110.00, 109.95.

HRMS m/z:[M]⁺ calcd for C₂₆H₁₈BrN, 423.0623. found, 423.0621.

### Synthesis Example 5: Synthesis of Intermediate Product I-5

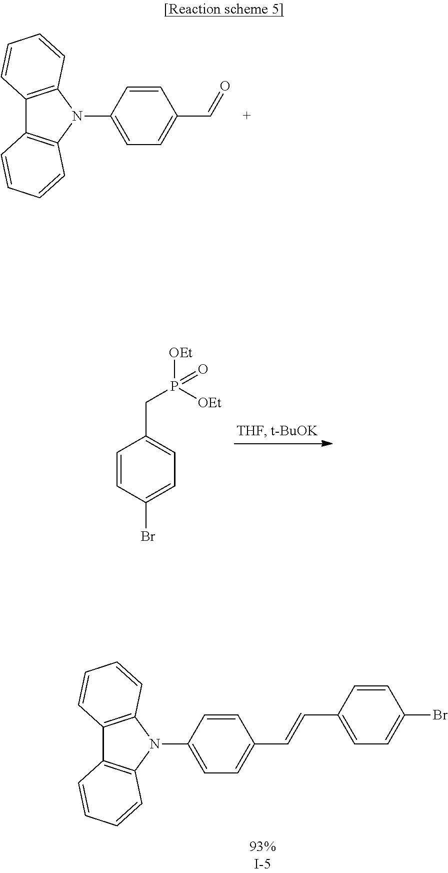

t-BuOK (0.22 g, 2 mmol) was placed in a two-neck bottle, and after the bottle was vacuumed and nitrogen was introduced, 3 mL of anhydrous tetrahydrofuran (THF) was added. 4-(9H-carbazol-9-yl)benzaldehyde (0.27 g, 1 mmol) and diethyl(4-bromobenzyl)phosphonate (0.34 g, 1.1 mmol) were placed in a single-neck bottle, and 3 mL of anhydrous tetrahydrofuran (THF) was added under a nitrogen atmosphere. The solution in the single-neck bottle was slowly added in a two-neck bottle in an ice bath to mix, and the mixture was reacted at 0° C. for 1 day. The reaction solution was poured into water to precipitate a yellow solid. Suction and filtering were performed on the precipitated yellow solid, then the precipitated yellow solid was cleaned repeatedly using methanol to obtain a light yellow powder intermediate product I-5 ((E)-9-(4-(4-bromostyryl)phenyl)-9H-carbazole) (0.39 g, yield: 93%).

¹H NMR (400 MHz, CDCl₃): δ 8.13 (d, J=7.6 Hz, 2H), 7.71 (d, J=8.4 Hz, 2H), 7.55 (d, J=8.4 Hz, 2H), 7.50 (d, J=8.8 Hz, 2H), 7.44-7.38 (m, 6H), 7.30-7.26 (m, 2H), 7.19 (d, J=16 Hz, 1H), 7.11 (d, J=16 Hz, 1H)

¹³C NMR (100 MHz, CDCl₃): δ 140.69, 137.09, 136.05, 13.01, 131.86, 128.34, 128.21, 128.04, 127.84, 127.19, 125.95, 123.42, 121.60, 120.32, 120.02, 109.77

### Synthesis Example 6: Synthesis of Intermediate Product I-6

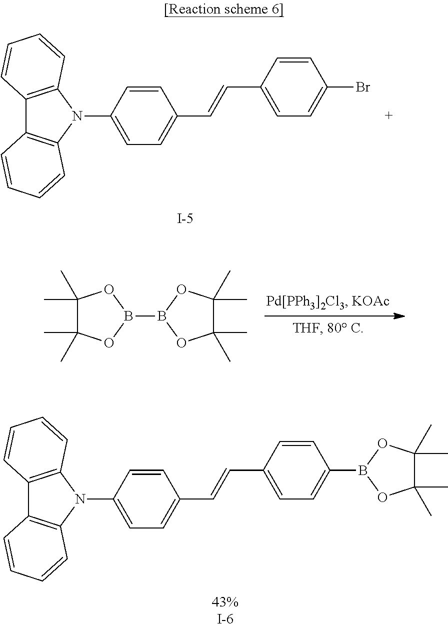

The intermediate product I-5 (0.42 g, 1 mmol) and 4,4,4′,4′,5,5,5′,5′-octamethyl-2,2′-bi(1,3,2-dioxaborolane) (0.31 g, 1.2 mmol) were placed in a high-pressure pipe. Potassium acetate (0.29 g, 2.93 mmol) and Pd(PPh₃)₂Cl₂ (0.04 g, 0.05 mmol) were added in a high-pressure pipe. 4 mL of anhydrous tetrahydrofuran (THF) was added in a nitrogen atmosphere, and the above compounds were mixed. The mixture was heated and reacted at 80° C. for 1 day, and then the reaction solution was filtered using celite and silica gel. After the solvent was removed via rotary concentration, purification was performed using column chromatography (ethyl acetate:n-hexane=1:5) to obtain a white intermediate product I-6 ((E)-9-(4-(4-(3,3,4,4-tetramethylborolan-1-yl)styryl)phenyl)-9H-carbazole) (0.20 g, yield: 43%).

¹H NMR (400 MHz, CDCl₃): δ8.14-8.12 (m, 2H), 7.82 (d, J=8.4 Hz, 2H), 7.73 (d, J=8.4 Hz, 2H), 7.55 (d, J=7.6 Hz, 4H), 7.44-7.38 (m, 4H), 7.30-7.25 (m, 3H), 7.19 (d, J=16.4 Hz, 1H), 1.35 (s, 12H)

¹³C NMR (100 MHz, CDCl₃): δ 140.72, 139.73, 136.98, 136.31, 135.21, 134.71, 129.45, 128.56, 127.89, 127.17, 125.95, 125.89, 123.40, 120.30, 119.98, 109.81, 83.82, 24.87

### Synthesis Example 7: Synthesis of Intermediate Product I-7

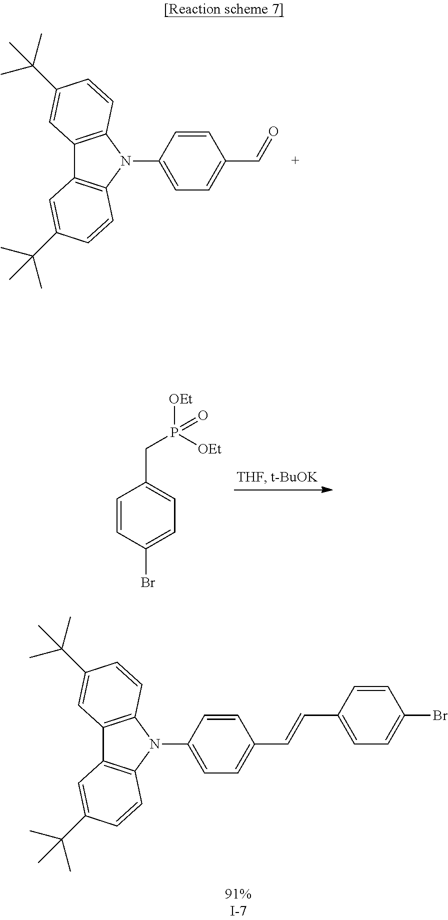

The intermediate product I-7 ((E)-9-(4-(4-bromostyryl)phenyl)-3,6-di-tert-butyl-9H-carbazole) was prepared using a method similar to synthesis example 5, and the difference thereof is only in that 4-(9H-carbazol-9-yl)benzaldehyde in synthesis example 5 was replaced with 4-(3,6-di-tert-butyl-9H-carbazol-9-yl)benzaldehyde (0.38 g, 1 mmol). A light yellow powder intermediate product I-7 (0.49 g, yield: 91%) was obtained according to the above method.

¹H NMR (400 MHz, CDCl₃): δ8.12 (d, J=1.2 Hz, 2H), 7.69 (d, J=8.4 Hz, 2H), 7.55-7.35 (m, 10H), 7.17 (d, J=16.4 Hz, 1H), 7.17 (d, J=16.4 Hz, 1H), 1.45 (s, 18H)

¹³C NMR (100 MHz, CDCl₃): δ142.96, 139.03, 137.65, 136.10, 135.52, 131.85, 128.47, 128.02, 127.93, 127.77, 126.77, 123.62, 123.42, 121.52, 116.25, 109.21

### Synthesis Example 8: Synthesis of Intermediate Product I-8

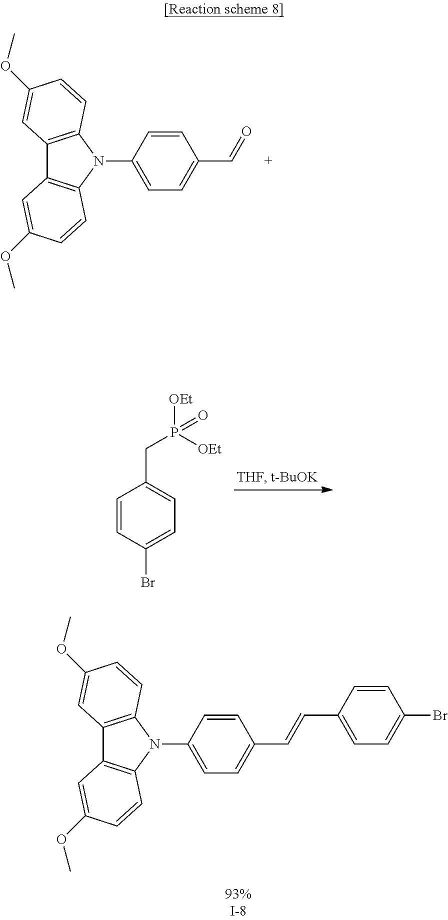

The intermediate product I-8 ((E)-9-(4-(4-bromostyryl)phenyl)-3,6-dimethoxy-9H-carbazole) was prepared using a method similar to synthesis example 5, and the difference thereof is only in that 4-(9H-carbazol-9-yl)benzaldehyde in synthesis example 5 was replaced with 4-(3,6-dimethoxy-9H-carbazol-9-yl)benzaldehyde (0.33 g, 1 mmol). A light yellow powder intermediate product I-7 (0.45 g, yield: 93%) was obtained according to the above method.

¹H NMR (400 MHz, CDCl₃): δ7.68 (d, J=8.0 Hz, 2H), 7.53-7.48 (m, 6H), 7.41-7.39 (m, 2H), 7.35 (d, J=8.8 Hz, 2H), 7.16 (d, J=16.4 Hz, 1H), 7.08 (d, J=16.4 Hz, 1H), 7.03 (dd, J=2.8, 9.2 Hz, 2H), 3.93 (s, 6H)

¹³C NMR (100 MHz, CDCl₃): δ154.06, 137.55, 136.03, 136.01, 135.44, 131.80, 128.35, 127.99, 127.90, 127.76, 126.60, 123.70, 121.49, 115.16, 110.71, 102.87

[Synthesis of Final Compound]

### Synthesis Example 9: Synthesis of Compound DPASP

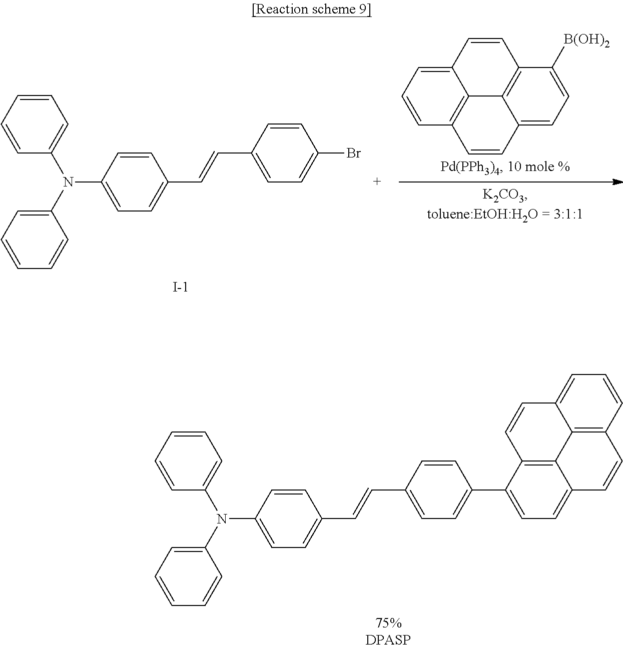 [SMILES: Brc1ccc(/C=C/c2ccc(N(c3ccccc3)c3ccccc3)cc2)cc1 ; OB(O)c1ccc2ccc3cccc4ccc1c2c34 ; [I-] ; C(=C\c1ccc(N(c2ccccc2)c2ccccc2)cc1)/c1ccc(-c2ccc3ccc4cccc5ccc2c3c45)cc1 ; "75%DPASP"]

The intermediate product I-1 (0.85 g, 2 mmol), 1-pyrenylboronic acid (0.59 g, 2.4 mmol), Pd(PPh₃)₄ (10 mg, 0.01 mmol), aqueous potassium carbonate solution (2.0 M, 3.5 mL), ethanol (3.5 mL), and toluene (10.5 mL) were placed in a two-neck bottle. Oxygen was removed and nitrogen was added, and the reaction was heated to 110° C. and stirred for 24 hours. The metal was filtered and removed and extracted via ethyl acetate (EA) and THF, and an organic layer was collected. Then, water was removed via magnesium sulfate (MgSO₄), and filtering was performed and the solvent was removed via concentration under reduced pressure. Then, purification was performed using column chromatography (dichloromethane:hexane=1:5), and solid was collected. Sublimation was performed at 265° C. to obtain a yellow compound DPASP ((E)-4-(4-(4,6-dihydropyren-1-yl)styryl)-N,N-diphenylaniline) (0.82 g, yield: 75%).

1H NMR (400 MHz, CDCl3, δ): 8.25-7.95 (m, 9H), 7.67 (d, J=8 Hz, 2H), 7.61 (d, J=8 Hz, 2H), 7.43 (d, J=8.4 Hz, 2H), 7.26 (dd, J=8.4 Hz, J=7.6 Hz, 4H), 7.18 (d, J=16.4 Hz, 1H), 7.12 (d, J=8.4 Hz, 2H), 7.11 (d, J=16.4 Hz, 1H), 7.07 (d, J=8.4 Hz, 4H), 7.01 (t, J=7.6 Hz, 2H).

¹³C NMR (100 MHz, CDCl₃, δ): 147.53, 147.46, 140.13, 137.40, 136.66, 131.49, 130.93, 130.58, 129.39, 128.49, 127.43, 126.60, 126.30, 126.01, 125.27, 125.10, 125.02, 124.93, 124.81, 124.68, 124.53, 123.57, 123.07.

HRMS m/z: [M]⁺ calcd for C₄₂H₂₉N, 547.2300. found, 547.2305.

Anal. calcd for C₄₂H₂₉N: C, 92.11, H, 5.34, N, 2.56. found: C, 91.89, H, 5.32, N, 2.47.

### Synthesis Example 10: Synthesis of Compound DFASP

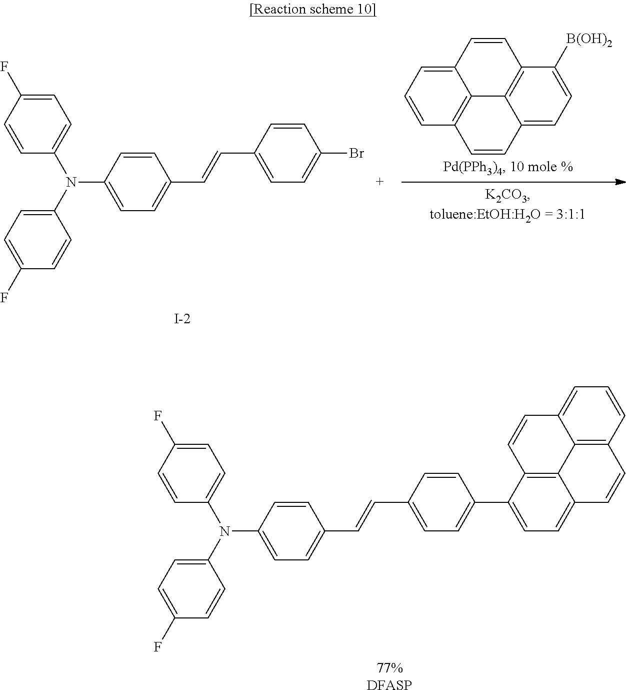

The intermediate product I-2 (0.92 g, 2 mmol), 1-pyrenylboronic acid (0.59 g, 2.4 mmol), Pd(PPh₃)₄ (10 mg, 0.01 mmol), aqueous potassium carbonate solution (2.0 M, 3.5 mL), ethanol (3.5 mL), and toluene (10.5 mL) were placed in a two-neck bottle. Oxygen was removed and nitrogen was added, and the reaction was heated to 110° C. and stirred for 24 hours. The metal was filtered and removed and extracted via ethyl acetate (EA) and THF, and an organic layer was collected. Then, water was removed via magnesium sulfate (MgSO₄), and filtering was performed and the solvent was removed via concentration under reduced pressure. Then, purification was performed using column chromatography (dichloromethane:hexane=1:5), and solid was collected. Sublimation was performed at 250° C. to obtain a yellow compound DFASP ((E)-4-(4-(4,6-dihydropyren-1-yl)styryl)-N,N-bis(4-fluorophenyl)aniline) (0.89 g, yield: 77%).

¹H NMR (400 MHz, CDCl₃, δ): 8.23-7.98 (m, 9H), 7.67 (d, J=8.4 Hz, 2H), 7.62 (d, J=8.4 Hz, 2H), 7.42 (d, J=8.8 Hz, 2H), 7.17 (d, J=16 Hz, 1H), 7.12-7.04 (m, 5H), 7.00-6.90 (m, 6H).

¹³C NMR (100 MHz, CDCl₃, δ): 158.00 ppm (d, ¹³C-¹⁹F coupling J=242 Hz, C), 147.44 (C), 143.50 (d, ¹³C-¹⁹F coupling J=2.3 Hz, C), 140.12 (C), 137.30 (C), 136.51 (C), 131.43 (C), 131.18 (C), 130.91 (CH), 130.53 (C), 128.39 (C), 128.28 (CH), 127.46 (CH), 127.38 (CH), 126.56 (CH), 126.27 (CH), 126.19 (CH), 126.11 (CH), 125.97 (CH), 125.19 (CH), 125.08 (CH), 124.97 (C), 124.87 (C), 124.79 (CH), 124.67 (CH), 122.27 (CH), 116.10 (d, ¹³C-¹⁹F coupling J=22.7 Hz, CH)

HRMS min/z: [M]⁺ calcd for C₄₂H₂₇F₂N, 583.2112. found, 583.2109.

Anal. calcd for C₄₂H₂₇F₂N: C, 86.43, H, 4.66, N, 2.40. found: C, 86.31, H, 4.70, N, 2.37.

### Synthesis Example 11: Synthesis of Compound NASP

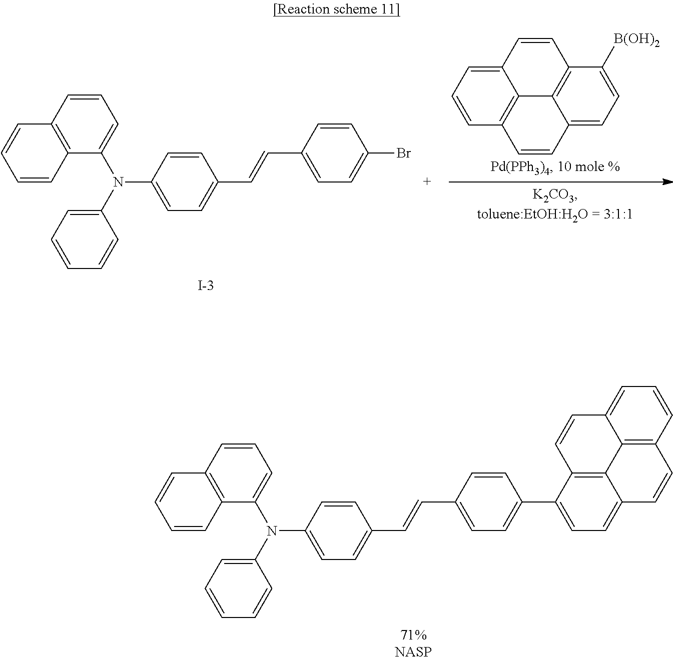

The intermediate product I-3 (0.95 g, 2 mmol), 1-pyrenylboronic acid (0.59 g, 2.4 mmol), Pd(PPh₃)₄ (10 mg, 0.01 mmol), aqueous potassium carbonate solution (2.0 M, 3.5 mL), ethanol (3.5 mL), and toluene (10.5 mL) were placed in a two-neck bottle. Oxygen was removed and nitrogen was added, and the reaction was heated to 110° C. and stirred for 24 hours. The metal was filtered and removed and extracted via ethyl acetate (EA), and an organic layer was collected. Then, water was removed via magnesium sulfate (MgSO₄), and filtering was performed and the solvent was removed via concentration under reduced pressure. Then, purification was performed using column chromatography (dichloromethane:hexane=1:5), and solid was collected. Sublimation was performed at 295° C. to obtain a yellow compound NASP ((E)-N-phenyl-N-(4-(4-(pyren-1-yl)styryl)phenyl)naphthalen-1-amine) (0.85 g, yield: 71%).

¹H NMR (400 MHz, CDCl₃, δ): 8.26-7.98 (m, 10H), 7.91 (d, J=8.0 Hz, 1H), 7.80 (d, J=8.0 Hz, 1H), 7.66-7.60 (m, 4H), 7.51-7.46 (m, 2H), 7.40-7.37 (m, 4H), 7.26-6.98 (m, 9H).

¹³C NMR (100 MHz, CDCl₃, δ): 141.23, 140.54, 139.83, 137.44, 137.37, 136.78, 131.40, 130.90, 130.46, 129.81, 129.69, 129.59, 128.36, 127.48, 127.41, 127.36, 127.31, 126.89, 126.21, 126.11, 125.93, 125.91, 125.23, 125.01, 124.95, 124.86, 124.74, 124.65, 123.74, 123.33, 120.37, 120.14, 118.61, 109.96, 109.91.

HRMSm/z:[M]⁺ calcd for C₄₆H₃₁N: 597.2457. found, 547.2456.

Anal. calcd for C₄₆H₃₁N: C, 92.43, H, 5.23, N, 2.34. found: C, 92.31, H, 5.20, N, 2.29.

### Synthesis Example 12: Synthesis of Compound PCzSP

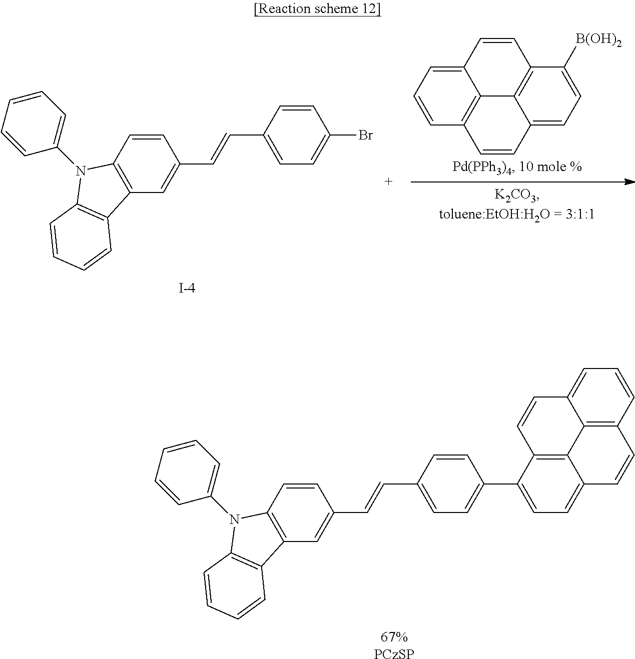

The intermediate product I-4 (0.85 g, 2 mmol), 1-pyrenylboronic acid (0.59 g, 2.4 mmol), Pd(PPh₃)₄ (10 mg, 0.01 mmol), aqueous potassium carbonate solution (2.0 M, 3.5 mL), ethanol (3.5 mL), and toluene (10.5 mL) were placed in a two-neck bottle. Oxygen was removed and nitrogen was added, and the reaction was heated to 110° C. and stirred for 24 hours. The metal was filtered and removed and extracted via ethyl acetate (EA), and an organic layer was collected. Then, water was removed via magnesium sulfate (MgSO₄), and filtering was performed and the solvent was removed via concentration under reduced pressure. Then, purification was performed using column chromatography (dichloromethane:hexane=1:5), and solid was collected. Sublimation was performed at 275° C. to obtain a yellow compound PCzSP ((E)-9-phenyl-3-(4-(pyren-1-yl)styryl)-9H-carbazole) (0.73 g, yield: 67%).

1H NMR (400 MHz, CDCl₃, δ): 8.33-7.99 (m, 11H), 7.75 (d, J=8.0 Hz, 2H), 7.68-7.57 (m, 7H), 7.50-7.40 (m, 5H), 7.36-7.27 (m, 2H).

¹³C NMR (100 MHz, CDCl₃, δ): 141.27, 140.58, 139.87, 137.47, 137.41, 136.81, 131.43, 130.91, 129.84, 129.71, 129.62, 128.40, 127.49, 127.46, 127.42, 127.37, 127.33, 126.94, 126.22, 126.12, 125.94, 125.25, 125.03, 124.97, 124.89, 124.75, 124.66, 123.76, 123.33, 120.38, 120.14, 118.61, 109.98, 109.92.

HRMS m/z:[M]⁺ calcd for C₄₂H₂₇N, 545.2143. found, 545.2138.

Anal. calcd for C₄₂H₂₇N: C, 92.45, H, 4.99, N, 2.57. found: C, 92.31, H, 5.04, N, 2.53.

### Synthesis Example 13: Synthesis of Compound CZSSO

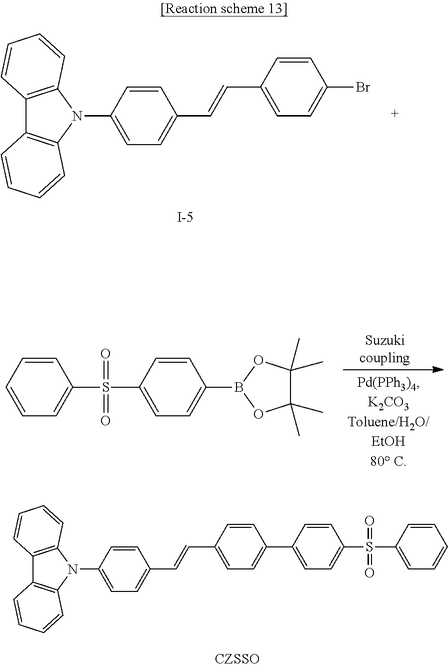

The intermediate product I-5 (0.42 g, 1 mmol) and 4,4,5,5-tetramethyl-2-(4-(phenylsulfonyl)phenyl)-1,3,2-dioxaborolane (0.34 g, 1 mmol) were placed in a high-pressure pipe, and potassium carbonate (0.49 g, 3.5 mmol) and Pd(PPh₃)₄ (0.12 g, 0.1 mmol) were added in the high-pressure pipe. Toluene (3 mL), water (1 mL), and ethanol (1 mL) were added in the high-pressure pipe under a nitrogen atmosphere, and the above compounds were mixed. The mixture was heated and reacted at 80° C. for 1 day, and then the reaction solution was filtered using celite and silica gel. After the solvent was removed via rotary concentration, purification was performed using column chromatography (dichloromethane:n-hexane=1:1) to obtain 0.49 g of a yellow solid (yield: 87%). Sublimation was performed at a temperature of 305° C. and a pressure of 9×10⁻⁶ torr to obtain a yellow compound CZSSO ((E)-9-(4-(2-(4′-(phenylsulfonyl)-[1,1′-biphenyl]-4-yl)vinyl)phenyl)-9H-carbazole) (yield: 82%).

¹H NMR (400 MHz, CDCl₃): δ 8.16 (d, J=7.6 Hz, 2H), 8.02-7.97 (m, 4H), 7.81-7.78 (m, 4H), 7.70-7.64 (m, 4H), 7.61-7.53 (m, 5H), 7.48-7.41 (m, 4H), 7.35-7.25 (m, 4H)

HRMS (m/z): [M⁺] calcd. for C₃₈H₂₇NO₂S, 561.1762. found, 561.1769.

Anal. calcd for C₃₈H₂₇NO₂S: C, 81.26; H, 4.85; N, 2.49. found: C, 81.34; H, 4.71; N, 2.55.

### Synthesis Example 14: Synthesis of Compound TCZSSO

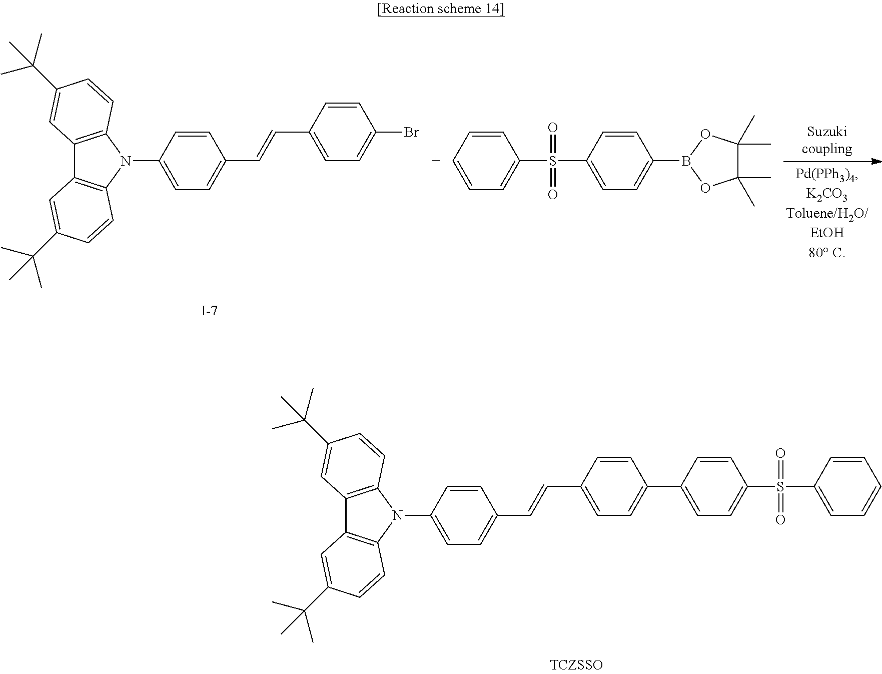

The intermediate product I-7 (0.54 g, 1 mmol) and 4,4,5,5-tetramethyl-2-(4-(phenylsulfonyl)phenyl)-1,3,2-dioxaborolane (0.34 g, 1 mmol) were placed in a high-pressure pipe, and potassium carbonate (0.49 g, 3.5 mmol) and Pd(PPh3)4 (0.12 g, 0.1 mmol) were added in the high-pressure pipe. Toluene (3 mL), water (1 mL), and ethanol (1 mL) were added in the high-pressure pipe under a nitrogen atmosphere, and the above compounds were mixed. The mixture was heated and reacted at 80° C. for 1 day, and then the reaction solution was filtered using celite and silica gel. After the solvent was removed via rotary concentration, purification was performed using column chromatography (dichloromethane:n-hexane=1:1) to obtain 0.56 g of a yellow solid (yield: 83%). Sublimation was performed at a temperature of 330° C. and a pressure of 9×10⁻⁶ torr to obtain a green glass-state compound TCZSSO ((E)-3,6-di-tert-butyl-9-(4-(2-(4′-(phenylsulfonyl)-[1,1′-biphenyl]-4-yl)vinyl)phenyl)-9H-carbazole) (yield: 84%).

¹H NMR (400 MHz, CDCl₃): δ 8.16 (d, J=1.6 Hz, 2H), 8.02-7.97 (m, 4H), 7.80-7.77 (m, 4H), 7.70-7.64 (m, 4H), 7.61-7.53 (m, 5H), 7.49 (dd, J=2, 8.8 Hz, 2H), 7.42-7.39 (m, 2H), 7.32 (d, J=16.4 Hz, 1H), 7.25 (d, J=16.4 Hz, 1H), 1.46 (s, 18H)

¹³C NMR (100 MHz, CDCl₃): δ 145.50, 142.97, 141.71, 140.07, 139.02, 138.20, 137.65, 137.56, 135.59, 133.17, 129.30, 128.70, 128.24, 128.21, 127.82, 127.64, 127.61, 127.19, 126.75, 123.61, 123.43, 116.24, 109.22, 34.71, 31.98

HRMS (m/z): [M⁺] calcd. for C₄₆H₄₃NO₂S, 673.3015. found, 673.3010.

Anal. calcd for C₄₆H₄₃NO₂S: C, 81.98; H, 6.43; N, 2.08. found: C, 81.87; H, 6.41; N, 2.13.

### Synthesis Example 15: Synthesis of Compound OCZSSO

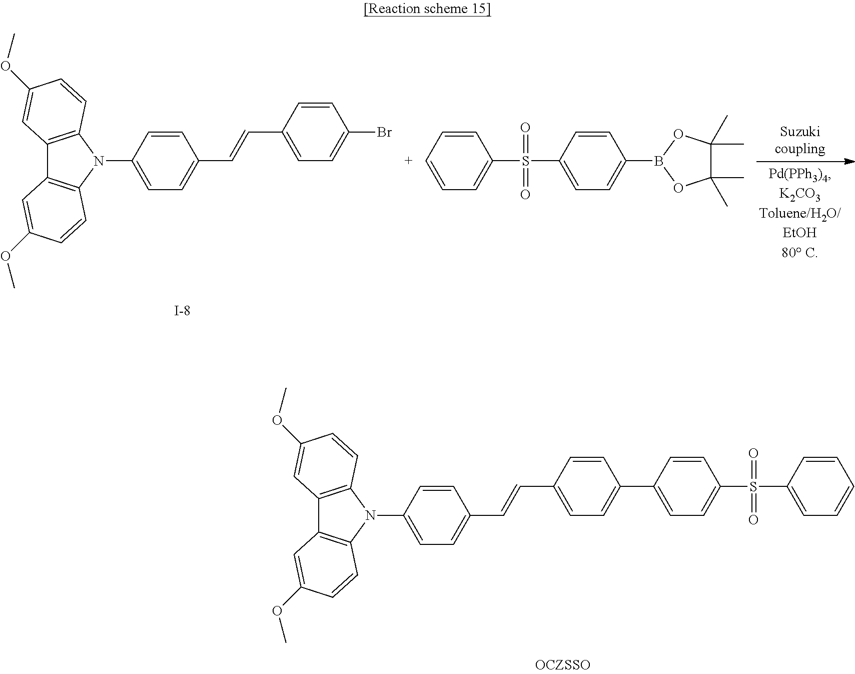

The intermediate product I-8 (0.48 g, 1 mmol) and 4,4,5,5-tetramethyl-2-(4-(phenylsulfonyl)phenyl)-1,3,2-dioxaborolane (0.34 g, 1 mmol) were placed in a high-pressure pipe, and potassium carbonate (0.49 g, 3.5 mmol) and Pd(PPh₃)₄ (0.12 g, 0.1 mmol) were added in the high-pressure pipe. Toluene (3 mL), water (1 mL), and ethanol (1 mL) were added in the high-pressure pipe under a nitrogen atmosphere, and the above compounds were mixed. The mixture was heated and reacted at 80° C. for 1 day, and then the reaction solution was filtered using celite and silica gel. After the solvent was removed via rotary concentration, purification was performed using column chromatography (dichloromethane:n-hexane=1:1) to obtain 0.52 g of a yellow solid (yield: 84%). Sublimation was performed at a temperature of 310° C. and a pressure of 9×10⁻⁶ torr to obtain a yellow glass-state compound OCZSSO ((E)-3,6-dimethoxy-9-(4-(2-(4′-(phenylsulfonyl)-[1,1′-biphenyl]-4-yl)vinyl)phenyl)-9H-carbazole) (yield: 76%).

¹H NMR (400 MHz, CDCl₃): δ 8.02-7.97 (m, 4H), 7.79-7.76 (m, 4H), 7.69-7.63 (m, 4H), 7.62-7.53 (m, 7H), 7.40-7.38 (m, 2H), 7.31 (d, J=16.4 Hz, 1H), 7.24 (d, J=16.4 Hz, 1H), 7.04 (dd, J=2.8, 9.2 Hz, 2H)

HRMS (m/z): [M⁺] calcd. for C₄₀H₃₁NO₄S, 621.1974. found, 621.1970.

Anal. calcd for C₄₀H₃₁NO₄S: C, 77.27; H, 5.03; N, 2.25. found: C, 77.11; H, 4.95; N, 2.31.

### Synthesis Example 16: Synthesis of Compound CZSDCN

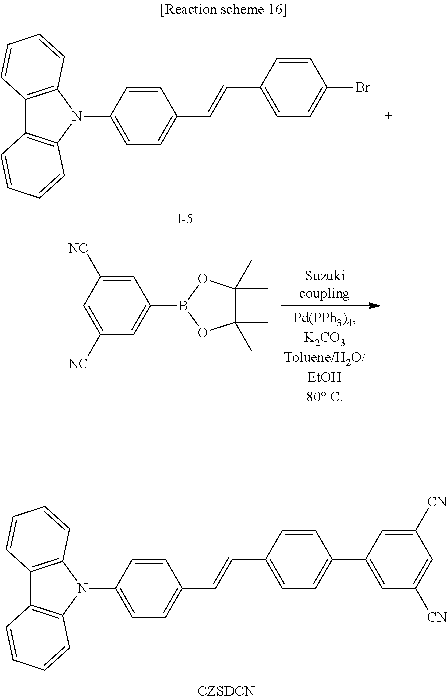

The intermediate product I-5 (0.42 g, 1 mmol) and 5-(4,4,5,5-tetramethyl-1,3,2-dioxaborolan-2-yl)isophthalonitrile (0.25 g, 1 mmol) were placed in a high-pressure pipe, and potassium carbonate (0.49 g, 3.5 mmol) and Pd(PPh₃)₄ (0.12 g, 0.1 mmol) were added in the high-pressure pipe. Toluene (3 mL), water (1 mL), and ethanol (1 mL) were added in the high-pressure pipe under a nitrogen atmosphere, and the above compounds were mixed. The mixture was heated and reacted at 80° C. for 1 day, and then the reaction solution was filtered using celite and silica gel. After the solvent was removed via rotary concentration, purification was performed using column chromatography (dichloromethane:n-hexane=2:1) to obtain 0.42 g of a yellow solid (yield: 89%). Sublimation was performed at a temperature of 290° C. and a pressure of 9×10⁻⁶ torr to obtain a yellow compound CZSDCN ((E)-4′-(4-(9H-carbazol-9-yl)styryl)-[1,1′-biphenyl]-3,5-dicarbonitrile) (yield: 83%).

¹H NMR (400 MHz, CDCl₃): δ 8.17-8.15 (m, 4H), 7.92 (t, J=1.4 Hz, 1H), 7.82 (d, J=8.4 Hz, 2H), 7.74 (d, J=8.4 Hz, 2H), 7.64-7.61 (m, 4H), 7.49-7.42 (m, 4H), 7.38-7.26 (m, 4H)

¹³C NMR (100 MHz, CDCl₃): δ 143.42, 140.66, 138.51, 137.36, 135.86, 135.52, 134.07, 133.32, 129.40, 128.02, 128.01, 127.61, 127.37, 127.22, 125.98, 123.46, 120.36, 120.09, 116.71, 114.65, 109.76

HRMS (m/z): [M⁺] calcd. for C₃₄H₂₁N₃, 471.1735. found, 471.1745.

Anal. calcd for C₃₄H₂₁N₃: C, 86.60; H, 4.49; N, 8.91. found: C, 86.39; H, 4.23; N, 9.21.

### Synthesis Example 17: Synthesis of Compound CZSDPT

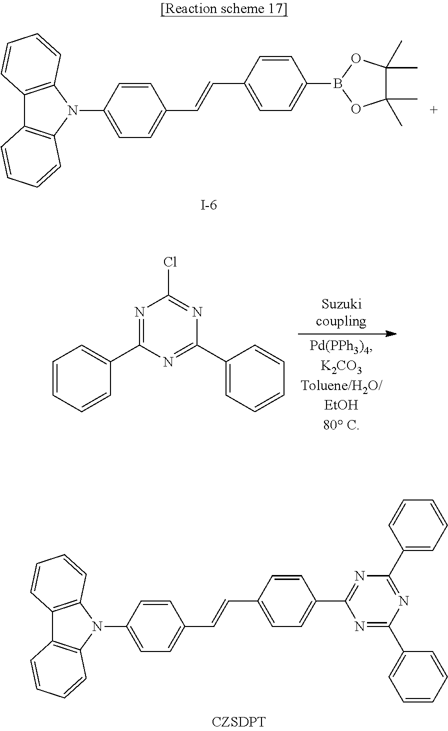

The intermediate product I-6 (0.47 g, 1 mmol) and 2-chloro-4,6-diphenyl-1,3,5-triazine (0.27 g, 1 mmol) were placed in a high-pressure pipe, and potassium carbonate (0.49 g, 3.5 mmol) and Pd(PPh₃)₄ (0.12 g, 0.1 mmol) were added in the high-pressure pipe. Toluene (3 mL), water (1 mL), and ethanol (1 mL) were added in the high-pressure pipe under a nitrogen atmosphere, and the above compounds were mixed. The mixture was heated and reacted at 80° C. for 1 day, and then the reaction solution was filtered using celite and silica gel. After the solvent was removed via rotary concentration, purification was performed using column chromatography (dichloromethane:n-hexane=1:1) to obtain 0.44 g of a yellow solid (yield: 76%). Sublimation was performed at a temperature of 310° C. and a pressure of 9×10⁻⁶ torr to obtain a yellow compound CZSDPT ((E)-9-(4-(4-(4,6-diphenyl-1,3,5-triazin-2-yl)styryl)phenyl)-9H-carbazole) (yield: 70%).

¹H NMR (400 MHz, CDCl₃): δ 8.84-8.80 (m, 5H), 8.17 (d, J=8.4 Hz, 2H), 7.83 (dd, J=8.4, 14.8 Hz, 4H), 7.68-7.60 (m, 8H), 7.51-7.42 (m, 6H), 7.38-7.29 (m, 3H)

¹³C NMR (100 MHz, CDCl₃): δ 171.58, 171.15, 141.14, 140.70, 137.28, 136.25, 136.06, 135.53, 132.49, 129.57, 129.43, 128.96, 128.84, 128.63, 128.67, 127.19, 126.76, 125.98, 123.46, 120.34, 120.05, 109.81

HRMS (m/z): [M⁺] calcd. for C₄₁H₂₈N₄, 576.2314. found, 576.2305.

Anal. calcd for C₄₁H₂₈N₄: C, 85.39; H, 4.89; N, 9.72. found: C, 85.07; H, 5.03; N, 9.60.

[Property Evaluation of Compounds]

[Luminescence Properties]

Table 1 shows the luminescence properties of the aromatic compounds of the above embodiments.

TABLE 1

| | | | | | Full | |
|---|---|---|---|---|---|---|
| | | Light | | Light | width | Quantum |
| | Absorption | emission | Absorption | emission | half | efficiency |
| | wavelength | wavelength | wavelength | wavelength | maximum | in |
| | in toluene | in toluene | in thin film | in thin film | in toluene | cyclohexane |
| Compound | (nm) | (nm) | (nm) | (nm) | (nm) | (%). |
| DPASP | 385 | 451 | — | 472 | 61 | >100 |
| DFASP | 381 | 444 | — | 480 | 57 | >100 |
| NASP | 382 | 447 | — | 473 | 61 | >100 |
| PCzSP | 361 | 434 | — | 458 | 59 | 85 |
| CZSSO | 293, 353 | 420 | 295, 366 | 460 | 57 | 127 |
| TCZSSO | 299, 363 | 430 | 299, 372 | 466 | 58 | 108 |
| OCZSSO | 311, 373 | 457 | 311, 380 | 497 | 68 | 126 |
| CZSDCN | 293, 358 | 427 | 295, 356 | 442 | 58 | 98 |
| CZSDPT | 286, 368 | 438 | 285, 381 | 473 | 61 | 103 |

It can be known from the results of Table 1 that, the fluorescent emission wavelength of the aromatic compounds of the above embodiments is distributed between 420 nm and 497 nm. In other words, the aromatic compounds of the above embodiments can emit blue light, and are therefore suitable as blue light-emitting materials. Moreover, the aromatic compound of the above embodiments also has high quantum efficiency.

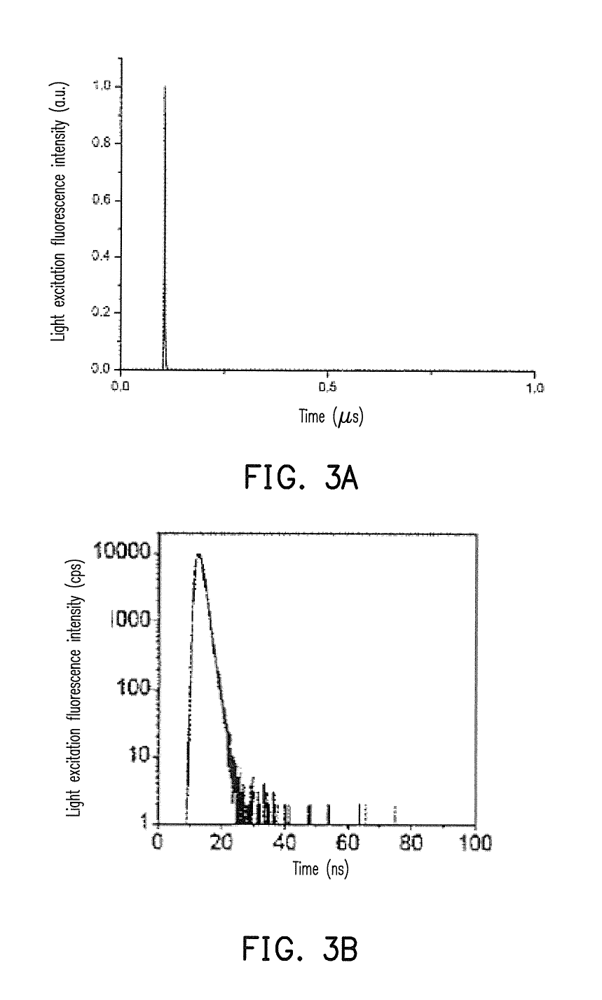

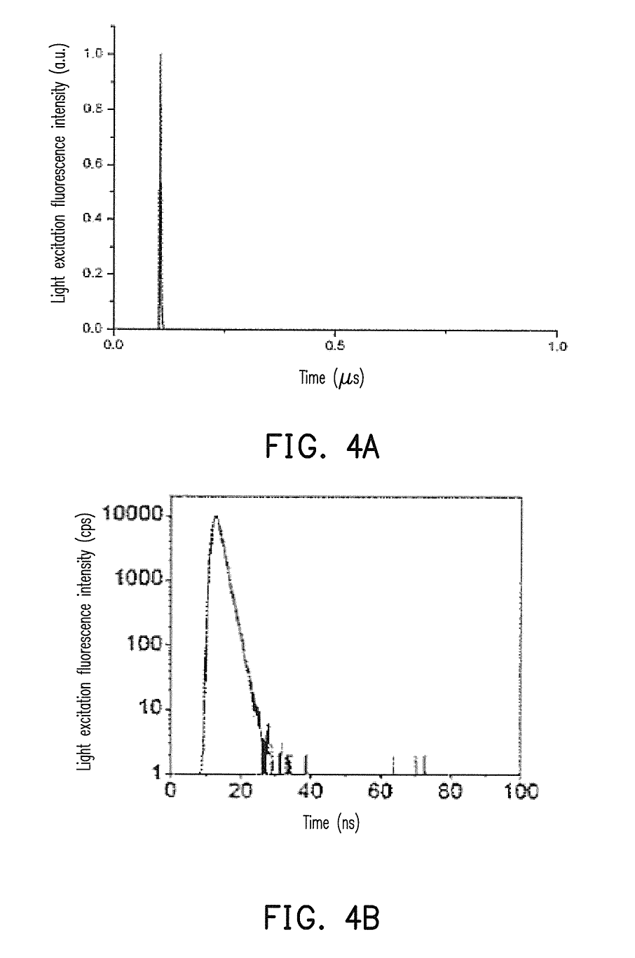

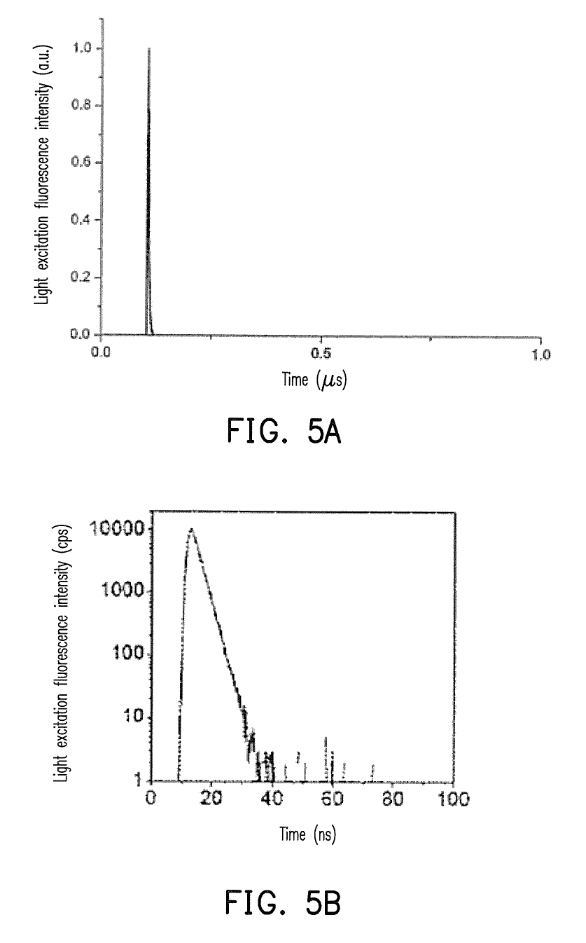

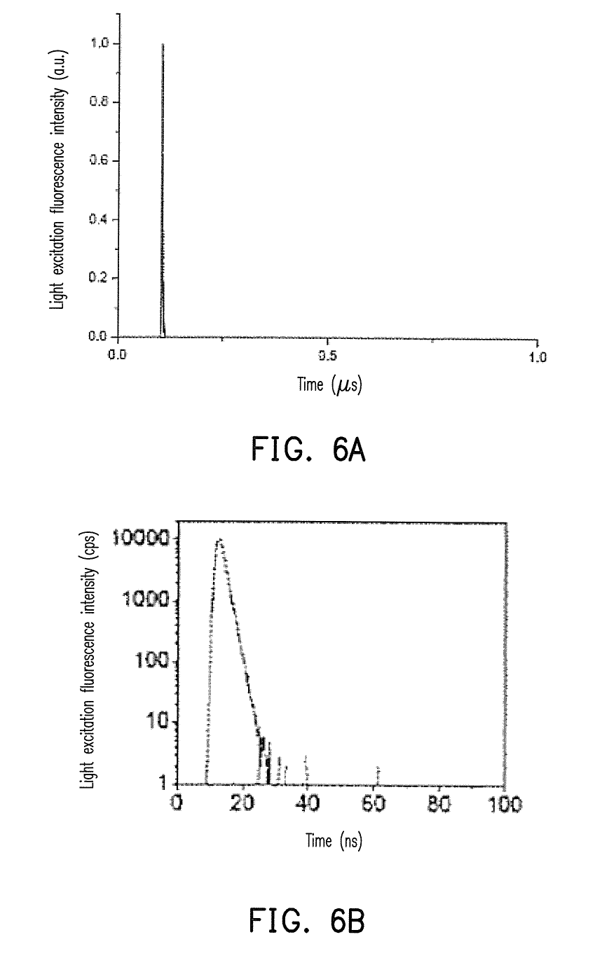

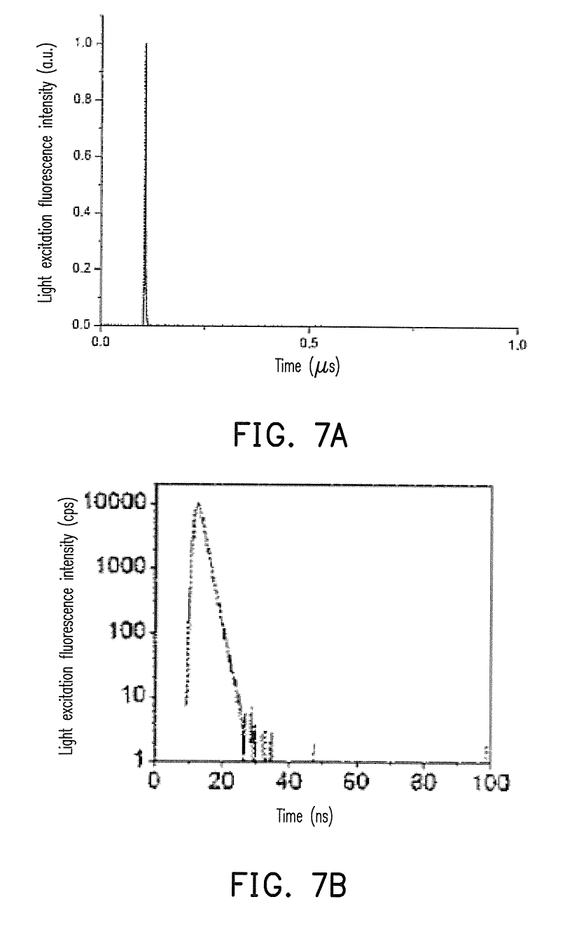

FIG. 3A and FIG. 3B show transient light excitation fluorescence curves of a toluene solution containing the compound CZSSO under the introduction of air and nitrogen, respectively. FIG. 4A and FIG. 4B show transient light excitation fluorescence curves of a toluene solution containing the compound TCZSSO under the introduction of air and nitrogen, respectively. FIG. 5A and FIG. 5B show transient light excitation fluorescence curves of a toluene solution containing the compound OCZSSO under the introduction of air and nitrogen, respectively. FIG. 6A and FIG. 6B show transient light excitation fluorescence curves of a toluene solution containing the compound CZSDCN under the introduction of air and nitrogen, respectively. FIG. 7A and FIG. 7B show transient light excitation fluorescence curves of a toluene solution containing the compound CZSDPT under the introduction of air and nitrogen, respectively.

In general, the organic light-emitting diode injects electric charge to the light-emitting substance from the anode and the cathode, and performs light emission via the resulting excitons in excited state. In the resulting excitons, 25% of the excitons are excited to singlet excited state, and the remaining 75% are excited to triplet excited state, wherein only excitons in singlet excited state can emit fluorescence. However, the specific light-emitting material has the characteristics of delayed fluorescence, and the source of fluorescence emission thereof is mainly from the transition of exciton radiation in triplet excited state to singlet excited state. Delayed fluorescence can be divided into triplet-triplet annihilation (TTA) delayed fluorescence and thermally-activated delayed fluorescence (TADF), wherein TTA delayed fluorescence relates to two excitons in triplet excited state converted to one exciton in singlet excited state capable of radiation transition via a collision annihilation process, such that a portion of the triplet excitons are partially reused; and the TADF delayed fluorescence relates to excitons in triplet excited state crossing over in reverse to singlet excited state via the absorption of heat energy to radiate fluorescence.

Currently, a known light-emitting material having TADF characteristics has delayed fluorescence phenomenon exceeding 500 ns in an aqueous solution. It can be known from the results of FIG. 3 to FIG. 7 that, regardless of whether air or nitrogen was introduced, the excitation of the thin film containing the aromatic compound (CZSSO, TCZSSO, OCZSSO, CZSDCN, CZSDPT) of the invention via light in an aqueous solution did not result in delayed fluorescence phenomenon. In other words, the aromatic compounds CZSSO, TCZSSO, OCZSSO, CZSDCN, and CZSDPT of the invention are not light-emitting materials having TADF characteristics.

[Thermal Stability Properties]

In the thermal stability test, thermal stability property testing was performed using a thermogravimetric differential thermal analyzer and a heating rate of 10° C./min to 20° C./min.

Table 2 shows the results of thermal stability testing of the aromatic compounds.

TABLE 2

| Compound | T_{g} (° C.) | T_{c} (° C.) | Tₘ (° C.) | T_{d} (° C.) |
|---|---|---|---|---|
| DPASP | 96 | N. D. | 270 | 439 |
| DFASP | N. D. | N. D. | 246 | 410 |
| NASP | 106 | N. D. | 226 | 452 |
| PCzSP | N. D. | N. D. | 204 | 431 |
| CZSSO | N. D. | N. D. | 285 | 407 |
| TCZSSO | 148 | 226 | 260 | 424 |
| OCZSSO | 111 | N. D. | 246 | 436 |
| CZSDCN | 147 | N. D. | 288 | 405 |
| CZSDPT | 76 | N. D. | 298 | 437 |

Tg: glass transition temperature; Tc: crystallization temperature; Tm: melting temperature; Td: thermal decomposition temperature; N. D.: not detected.

It can be known from the results of Table 2 that, the thermal decomposition temperatures of the aromatic compounds of the present application are all higher than 400° C., and the aromatic compounds of the present application all have excellent thermal stability.

[Manufacture of Organic Light-Emitting Diode]

### Experimental Example 1

DMPPP was used as the host light-emitting material, and the compound DPASP obtained in synthesis example 9 was used as the guest light-emitting material (i.e., dopant) to manufacture the organic light-emitting diode.

Specifically, the manufacturing process of the organic light-emitting diode is as shown below: first, N,N′-di(naphthalen-1-yl)-N,N′-diphenylbiphenyl-4,4′-diamine (NPB) (60 nm) and NPB (10 nm) doped with 3% of the compound DPASP were deposited on an ITO glass substrate (150 nm) used as the anode in order to form a hole transport layer. Then, the host light-emitting material DMPPP (15 nm) doped with 5% of the compound DPASP was deposited on the hole transport layer to form a light-emitting layer. Then, bis(2-methyl-8-quinolinolate)-4-(phenylphenolato)aluminium (BAlq) (20 nm) was deposited on the light-emitting layer to form an electron transport layer. Then, LiF (1 nm) and Al (100 nm) were deposited on the electron transport layer in order to form a cathode. At this point, the manufacture of the organic light-emitting diode of the present experimental example was complete. The above organic light-emitting diode has the following structure: ITO/NPB (60 nm)/NPB:3% DPASP (10 nm)/DMPPP: 3% DPASP (15 n)/BAlq (20 nm)/LiF (1 nm)/Al (100 nm).

### Experimental Example 2

The organic light-emitting diode was formed using a similar method to experimental example 1, and the difference thereof is only in that the compound DFASP obtained in synthesis example 10 was used as the dopant of the hole transport layer and the light-emitting layer.

### Experimental Example 3

The organic light-emitting diode was formed using a similar method to experimental example 1, and the difference thereof is only in that the compound NASP obtained in synthesis example 11 was used as the dopant of the hole transport layer and the light-emitting layer.

### Experimental Example 4

The organic light-emitting diode was formed using a similar method to experimental example 1, and the difference thereof is only in that the compound PCzSP obtained in synthesis example 12 was used as the dopant of the hole transport layer and the light-emitting layer.

### Experimental Example 5

CBP was used as the host light-emitting material, and the compound DPASP obtained in synthesis example 9 was used as the guest light-emitting material (i.e., dopant) to manufacture the organic light-emitting diode.

Specifically, the manufacturing process of the organic light-emitting diode is as shown below: first, NPB (30 nm) and 4,4′,4″-tri(N-carbazolyl)triphenylamine (TCTA) (20 nm) were deposited on an ITO glass substrate (150 nm) used as the anode in order to form a hole transport layer. Then, the host light-emitting material CBP (30 nm) doped with 3% of the compound DPASP was deposited on the hole transport layer to form a light-emitting layer. Then, 1,3,5-tris [(3-pyridyl)-3-phenyl] benzene (TmPyPb) (30 nm) was deposited on the light-emitting layer to form an electron transport layer. Then, LiF (1 nm) and Al (100 nm) were deposited on the electron transport layer in order to form a cathode. At this point, the manufacture of the organic light-emitting diode of the present experimental example was complete. The above organic light-emitting diode has the following structure: ITO/NPB (30 nm)/TCTA ((20 nm)/CBP: 3% DPASP (30 nm)/TmPyPb (30 nm)/LiF (1 nm)/Al (100 nm).

### Experimental Example 6

The organic light-emitting diode was formed using a similar method to experimental example 5, and the difference thereof is only in that the concentration of the dopant compound DPASP was 5%.

### Experimental Example 7

The organic light-emitting diode was formed using a similar method to experimental example 5, and the difference thereof is only in that the concentration of the dopant compound DPASP was 10%.

### Experimental Example 8

The organic light-emitting diode was formed using a similar method to experimental example 5, and the difference thereof is only in that the compound DFASP obtained in synthesis example 10 was used as the dopant of the light-emitting layer, and the concentration of the compound DFASP was 5%.

### Experimental Example 9

The organic light-emitting diode was formed using a similar method to experimental example 5, and the difference thereof is only in that the compound NASP obtained in synthesis example 11 was used as the dopant of the light-emitting layer, and the concentration of the compound NASP was 5%.

### Experimental Example 10

The organic light-emitting diode was formed using a similar method to experimental example 5, and the difference thereof is only in that the compound PCzSP obtained in synthesis example 12 was used as the dopant of the light-emitting layer, and the concentration of the compound PCzSP was 5%.

### Experimental Example 11

DMPPP was used as the host light-emitting material, and the compound CZSSO obtained in synthesis example 13 was used as the guest light-emitting material (i.e., dopant) to manufacture the organic light-emitting diode.

Specifically, the manufacturing process of the organic light-emitting diode is as shown below: first, NPB (10 nm) and TCTA (40 nm) were deposited on an ITO glass substrate (150 nm) used as the anode in order to form a hole transport layer. Then, the host light-emitting material DMPPP (30 nm) doped with 10% of the compound CZSSO was deposited on the hole transport layer to form a light-emitting layer. Then, TmPyPb (40 nm) was deposited on the light-emitting layer to form an electron transport layer. Then, LiF (1 nm) and Al (100 nm) were deposited on the electron transport layer in order to form a cathode. At this point, the manufacture of the organic light-emitting diode of the present experimental example was complete. The above organic light-emitting diode has the following structure: ITO/NPB (10 nm)/TCTA ((40 nm)/DMPPP: 10% CZSSO (30 nm)/TmPyPb (40 nm)/LiF (1 nm)/Al (100 nm).

### Experimental Example 12

The organic light-emitting diode was formed using a similar method to experimental example 11, and the difference thereof is only in that the compound TCZSSO obtained in synthesis example 14 was used as the dopant of the light-emitting layer.

### Experimental Example 13

The organic light-emitting diode was formed using a similar method to experimental example 11, and the difference thereof is only in that the compound OCZSSO obtained in synthesis example 15 was used as the dopant of the light-emitting layer.

### Experimental Example 14

The organic light-emitting diode was formed using a similar method to experimental example 11, and the difference thereof is only in that the compound CZSDCN obtained in synthesis example 16 was used as the dopant of the light-emitting layer.

### Experimental Example 15

The organic light-emitting diode was formed using a similar method to experimental example 11, and the difference thereof is only in that the compound CZSDPT obtained in synthesis example 17 was used as the dopant of the light-emitting layer.

### Experimental Example 16

The organic light-emitting diode was formed using a similar method to experimental example 11, and the difference thereof is only in that CBP was used as the host light-emitting material, and the concentration of the compound CZSSO was 7%.

### Experimental Example 17

The organic light-emitting diode was formed using a similar method to experimental example 11, and the difference thereof is only in that CBP was used as the host light-emitting material, and the compound TCZSSO obtained in synthesis example 14 was used as the dopant of the light-emitting layer, wherein the concentration of the compound TCZSSO was 7%.

### Experimental Example 18

The organic light-emitting diode was formed using a similar method to experimental example 11, and the difference thereof is only in that CBP was used as the host light-emitting material, and the compound OCZSSO obtained in synthesis example 15 was used as the dopant of the light-emitting layer, wherein the concentration of the compound OCZSSO was 7%.

### Experimental Example 19

The organic light-emitting diode was formed using a similar method to experimental example 11, and the difference thereof is only in that CBP was used as the host light-emitting material, and the compound CZSDCN obtained in synthesis example 16 was used as the dopant of the light-emitting layer, wherein the concentration of the compound CZSDCN was 7%.

### Experimental Example 20

The organic light-emitting diode was formed using a similar method to experimental example 11, and the difference thereof is only in that CBP was used as the host light-emitting material, and the compound CZSDPT obtained in synthesis example 17 was used as the dopant of the light-emitting layer, wherein the concentration of the compound CZSDPT was 7%.

### Comparative Example

The organic light-emitting diode was formed using a similar method to experimental example 5, and the difference thereof is only in that the light-emitting layer does not have a dopant.

[Effectiveness Evaluation of Organic Light-Emitting Diode]

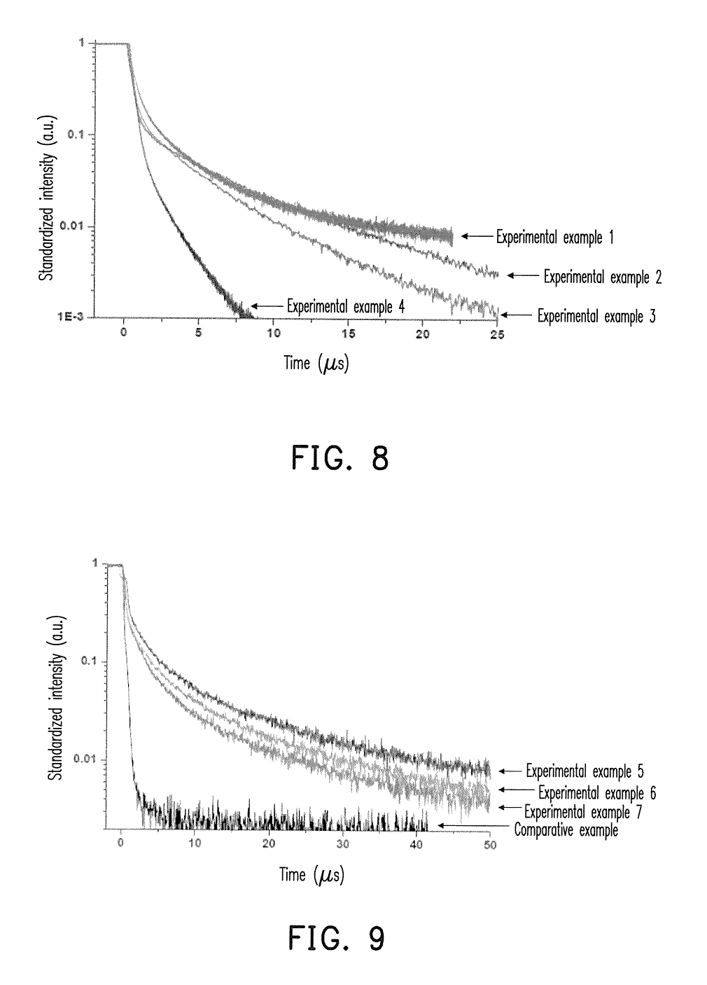

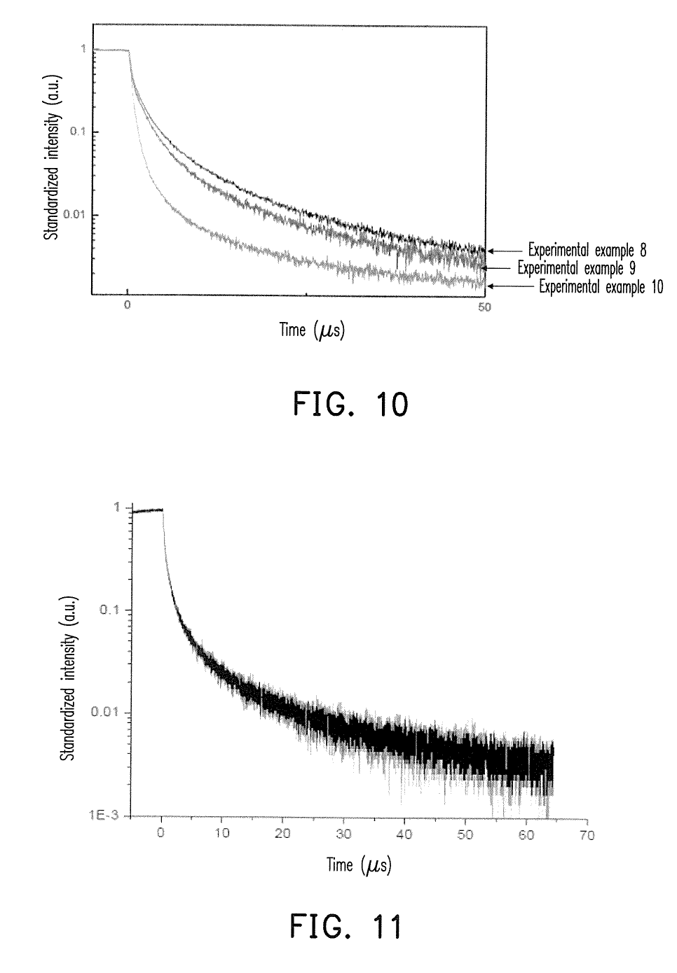

FIG. 8 shows transient light excitation fluorescence curves of the organic light-emitting diodes of experimental example 1 to experimental example 4. FIG. 9 shows transient light excitation fluorescence curves of the organic light-emitting diodes of experimental example 5 to experimental example 7 and the comparative example. FIG. 10 shows transient light excitation fluorescence curves of the organic light-emitting diodes of experimental example 8 to experimental example 10. FIG. 11 shows a transient light excitation fluorescence curve of the organic light-emitting diode of experimental example 18.

It can be known from the results of FIG. 8 to FIG. 11 that, in comparison to the light-emitting diode of the comparative example without a delayed fluorescence phenomenon, experimental example 1 to experimental example 10 and experimental example 18 have a delayed fluorescence phenomenon.

It should be mentioned here that, since it is known from the results of FIG. 5A and FIG. 5B that the aromatic compound OCZSSO is not a light-emitting material having TADF characteristics, it can be known that the delayed fluorescence characteristics shown by the organic light-emitting diode of experimental example 18 come from TTA delayed fluorescence.

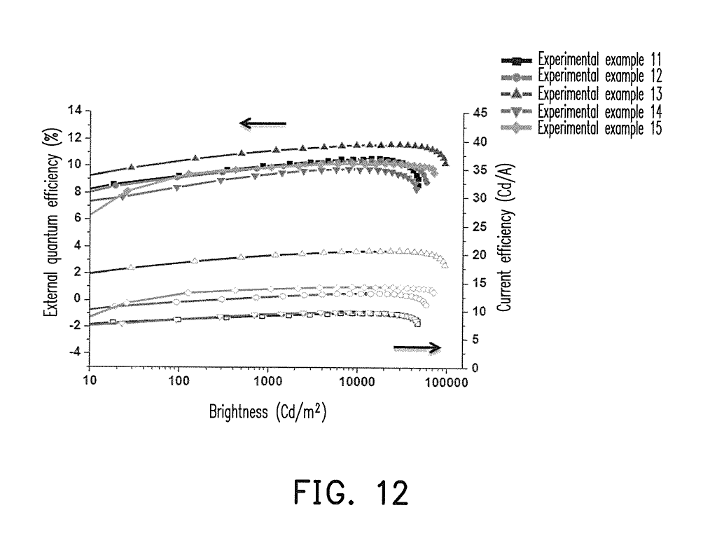

FIG. 12 shows brightness-external quantum efficiency curves of the organic light-emitting diodes of experimental example 11 to experimental example 15.

It can be known from the results of FIG. 12 that, the external quantum efficiency of the organic light-emitting diodes of experimental example 11 to experimental example 15 is not reduced with increase in brightness, indicating that the organic light-emitting diodes of experimental example 11 to experimental example 15 have the characteristics of long life.

Table 3 is the results of the effectiveness of the organic light-emitting diodes of experimental example 1 to experimental example 20 and the comparative example.

TABLE 3

| | | | | | | | | | Maximum |
|---|---|---|---|---|---|---|---|---|---|
| | Host | | | | Lₘₐₓ | C.E.. | P.E. | | radiation |
| | light-emitting | Dopant | V_{d} | E.Q.E. | (cd/m², | (cd/A, | (lm/W, | CIE | wavelength |
| | material | (%) | (V) | (%, V) | V) | V) | V) | (x, y) | (nm) |
| Experimental | DMPPP | DPASP | 2.57 | 10.7, | 68213, | 13.0, 5.0 | 8.9, 4.0 | (0.14, | 458 |
| example 1 | | | | 5.0 | 20.0 | | | 0.14) | |
| Experimental | DMPPP | DFASP | 2.92 | 10.9, | 58963, | 11.9, | 6.1, 6.0 | (0.14, | 456 |
| example 2 | | | | 7.5 | 17.5 | 7.5 | | 0.12) | |
| Experimental | DMPPP | NASP | 2.89 | 9.3, | 58263, | 10.2, 9.0 | 4.5, 6.0 | (0.14, | 456 |
| example 3 | | | | 9.0 | 18.5 | | | 0.12) | |
| Experimental | DMPPP | PCzSP | 2.98 | 7.7, | 34789, | 7.0, | 3.5, 5.5 | (0.15, | 446 |
| example 4 | | | | 8.0 | 18.5 | 8.0 | | 0.10) | |
| Experimental | CBP | DPASP | 3.1 | 10.8, | 38275, | 14.7, 3.5 | 13.2, 3.5 | (0.14, | 457 |
| example 5 | | | | 3.5 | 20.0 | | | 0.17) | |
| Experimental | CBP | DPASP | 3.0 | 12.0, | 42656, | 18.5, 3.5 | 16.6, 3.5 | (0.14, | 461 |
| example 6 | | | | 3.5 | 20.0 | | | 0.20) | |
| Experimental | CBP | DPASP | 3.0 | 10.6, | 48493, | 17.3, 3.5 | 15.5, 3.5 | (0.14, | 462 |
| example 7 | | | | 3.5 | 20.0 | | | 0.21) | |
| Experimental | CBP | DFASP | 3.06 | 11.9, | 30205, | 14.4, 3.5 | 12.9, 3.5 | (0.14, | 456 |
| example 8 | | | | 3.5 | 18.5 | | | 0.14) | |
| Experimental | CBP | NASP | 3.03 | 10.0, | 34729, | 14.1, 3.5 | 12.7, 3.5 | (0.14, | 462 |
| example 9 | | | | 3.5 | 18.5 | | | 0.17) | |
| Experimental | CBP | PCzSP | 3.31 | 10.0, | 25048, | 8.9, | 8.1, | (0.15, | 462 |
| example | | | | 3.5 | 20.0 | 3.5 | 3.5 | 0.09) | |
| 10 | | | | | | | | | |
| Experimental | DMPPP | CZSSO | 3.3 | 10.6, | 47449, | 9.6, | 6.1, | (0.15, | 444 |
| example | | | | 8.5 | 14.5 | 9.0 | 4.0 | 0.10) | |
| 11 | | | | | | | | | |
| Experimental | DMPPP | TCZSSO | 3.2 | 10.4, | 58609, | 13.1, 9.5 | 8.3, | (0.15, | 466 |
| example | | | | 8.0 | 14.5 | | 4.0 | 0.15) | |
| 12 | | | | | | | | | |
| Experimental | DMPPP | OCZSSO | 3.2 | 11.6, | 94762, | 20.6, 9.5 | 13.7, 3.5 | (0.15, | 476 |
| example | | | | 7.5 | 14.0 | | | 0.24) | |
| 13 | | | | | | | | | |
| Experimental | DMPPP | CZSDCN | 3.5 | 9.8, | 44983, | 9.7, | 5.5, | (0.15, | 450 |
| example | | | | 8.5 | 14.0 | 8.5 | 4.0 | 0.11) | |
| 14 | | | | | | | | | |
| Experimental | DMPPP | CZSDPT | 3.5 | 10.2, | 71114, | 14.2, 9.5 | 8.2, | (0.15, | 468 |
| example | | | | 7.5 | 14.0 | | 5.0 | 0.17) | |
| 15 | | | | | | | | | |
| Experimental | CBP | CZSSO | 3.6 | 9.7, | 8383, | 6.6, | 5.2, | (0.15, | 440 |
| example | | | | 4.0 | 15.0 | 4.0 | 4.0 | 0.07) | |
| 16 | | | | | | | | | |
| Experimental | CBP | TCZSSO | 3.6 | 8.0, | 12619, | 7.2, | 5.6, | (0.14, | 448 |
| example | | | | 4.0 | 13.5 | 4.0 | 4.0 | 0.10) | |
| 17 | | | | | | | | | |
| Experimental | CBP | OCZSSO | 3.6 | 9.2, | 19579, | 12.5, 4.0 | 9.8, | (0.14, | 462 |
| example | | | | 4.0 | 14.0 | | 4.0 | 0.17) | |
| 18 | | | | | | | | | |
| Experimental | CBP | CZSDCN | 3.8 | 8.8, | 9954, | 6.1, | 4.8, | (0.15, | 440 |
| example | | | | 4.0 | 13.5 | 4.0 | 4.0 | 0.08) | |
| 19 | | | | | | | | | |
| Experimental | CBP | CZSDPT | 3.9 | 9.1, | 11073, | 8.6, | 6.8, | (0.14, | 448 |
| example | | | | 4.0 | 14.5 | 4.0 | 4.0 | 0.10) | |
| 20 | | | | | | | | | |
| Comparative | CBP | None | 3.88 | 1.9, | 3460, | 1.0, | 0.6, | (0.17, | 400 |
| examples | | | | 5.5 | 16.5 | 5.5 | 4.5 | 0.11) | |

V_{d}: driving voltage;
E.Q.E: external quantum efficiency;
Lₘₐₓ: maximum brightness;
C.E.: current efficiency;
P.E.: power efficiency;
CIE: chromaticity coordinates

It can be known from the results of Table 3 that, the maximum radiation wavelength of the organic light-emitting diodes of experimental example 1 to experimental example 20 is located in the range of 440 nm to 476 nm, and therefore the organic light-emitting diodes of experimental example 1 to experimental example 20 have the characteristics of blue light emission.

Moreover, in comparison to the organic light-emitting diode of the comparative example without a dopant in the light-emitting layer, since the light-emitting layer of the organic light-emitting diodes of experimental example 1 to experimental example 20 has the aromatic compound of the invention, the organic light-emitting diodes of experimental example 1 to experimental example 20 have significantly higher external quantum efficiency, maximum brightness, current efficiency, and power efficiency.

Based on the above, the aromatic compound of the invention has the characteristics of blue light emission, high quantum efficiency, and good thermal stability. Moreover, the aromatic compound of the invention can be doped in the light-emitting layer or the hole transport layer of an organic light-emitting diode to increase external quantum efficiency, maximum brightness, current efficiency, power efficiency, and life of the organic light-emitting diode.

Although the invention has been described with reference to the above embodiments, it will be apparent to one of ordinary skill in the art that modifications to the described embodiments may be made without departing from the spirit of the invention. Accordingly, the scope of the invention is defined by the attached claims not by the above detailed descriptions.

What is claimed is:

1. An aromatic compound represented by the following chemical formula 1: 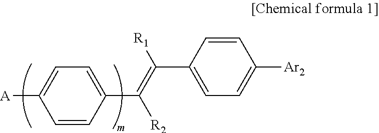
in chemical formula 1,
R₁ and R₂ are each independently hydrogen, halogen, a C₁ to C₆ alkyl group, or an aryl group;
m is an integer of 0 or 1;
A is a substituted or unsubstituted carbazole group Ar₁ or an organic amine group; and
Ar₂ is a substituted or unsubstituted pyrenyl group, a substituted or unsubstituted sulfonyl group, a substituted or unsubstituted triazine group, or a substituted or unsubstituted

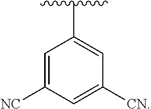 [SMILES: *c1cc(C#N)cc(C#N)c1]

2. The aromatic compound of claim 1, wherein the aromatic compound is represented by the following chemical formula 2: 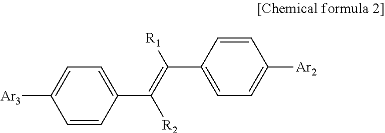
in chemical formula 2,
Ar₃ is selected from the following structural formulas, 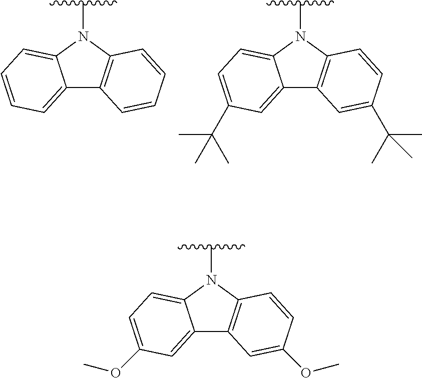
the remaining substituents are defined the same as in chemical formula 1.

3. The aromatic compound of claim 1, wherein the aromatic compound is represented by the following chemical formula 3: 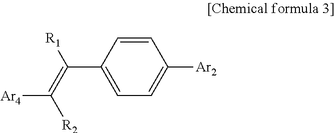
in chemical formula 3,
Ar₄ is selected from the following structural formulas, 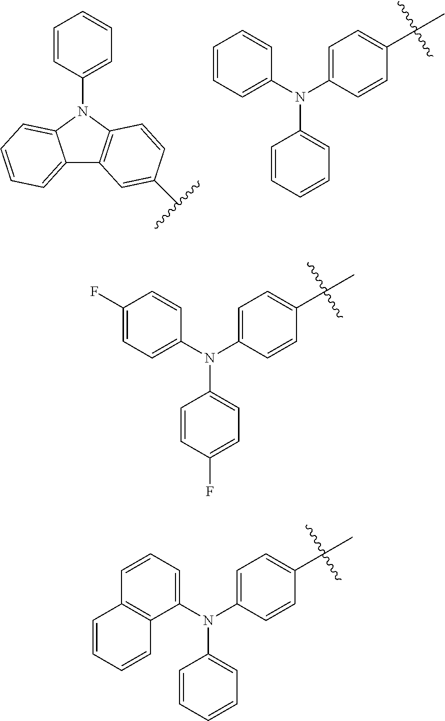 [SMILES: *c1ccc2c(c1)c1ccccc1n2-c1ccccc1 ; *c1ccc(N(c2ccccc2)c2ccccc2)cc1 ; *c1ccc(N(c2ccc(F)cc2)c2ccc(F)cc2)cc1 ; *c1ccc(N(c2ccccc2)c2cccc3ccccc23)cc1]
the remaining substituents are defined the same as in chemical formula 1.

4. The aromatic compound of claim 1, wherein Ar₂ is selected from the following structural formulas,

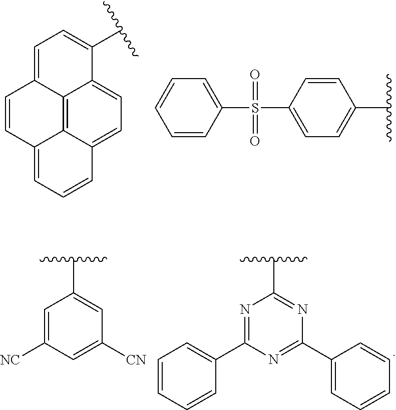

5. An organic light-emitting diode, comprising:
a cathode;
an anode; and
a light-emitting layer disposed between the cathode and the anode, wherein the light-emitting layer contains the aromatic compound of claim 1.

6. The organic light-emitting diode of claim 5, wherein the organic light-emitting diode is a blue light-emitting diode.

7. The organic light-emitting diode of claim 5, wherein the light-emitting layer comprises a host light-emitting material and a guest light-emitting material.

8. The organic light-emitting diode of claim 7, wherein the host light-emitting material comprises the aromatic compound.

9. The organic light-emitting diode of claim 7, wherein the guest light-emitting material comprises the aromatic compound.

10. The organic light-emitting diode of claim 7, wherein the host light-emitting material comprises 1-(2,5-dimethyl-4-(1-pyrenyl) phenyl)pyrene (DMPPP), 4,4′-N,N′-dicarbazole-biphenyl (CBP), or 2-(3-(pyren-1-yl)phenyl)triphenylene (m-PPT).

11. The organic light-emitting diode of claim 5, further comprising at least one auxiliary layer, and the auxiliary layer is selected from the group consisting of a hole injection layer, a hole transport layer, a hole blocking layer, an electron injection layer, an electron transport layer, and an electron blocking layer.

12. The organic light-emitting diode of claim 11, wherein the at least one auxiliary layer comprises the aromatic compound of claim 1.
